SEQUENCE LISTING (70 sequences)

SEQ ID NO: 1 - protein, 22 aa, Homo sapiens
MLLLVTSLLLCELPHPAFLLIP

SEQ ID NO: 2 - protein, 21 aa, Homo sapiens
MALPVTALLLPLALLLHAARP

SEQ ID NO: 3 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 4 - protein, 88 aa, Homo sapiens
PKPSISSNNSKPVEDKDAVAFTCEPEAQNTTYLWWVNGQSLPVSPRLQLSNGNRTLTLFNVTRNDARAYVCGIQNSVSANRSDPVTLD

SEQ ID NO: 5 - protein, 254 aa, Synthetic construct
DIVLTQSPASLAVSLGQRATMSCRAGESVDIFGVGFLHWYQQKPGQPPKLLIYRASNLESGIPVRFSGTGSRTDFTLIIDPVEADDVATYYCQQTNEDPYTFGGGTKLEIKGSTSGGGSGGGSGGGGSSEVQLQQSGAELVEPGASVKLSCTASGFNIKDTYMHWVKQRPEQGLEWIGRIDPANGNSKYVPKFQGKATITADTSSNTAYLQLTSLTSEDTAVYYCAPFGYYVSDYAMAYWGQGTSVTVSSTTTK

SEQ ID NO: 6 - protein, 111 aa, Synthetic construct
DIQLTQSPSSLSASVGDRVTITCRAGESVDIFGVGFLHWYQQKPGKAPKLLIYRASNLESGVPSRFSGSGSRTDFTLTISSLQPEDFATYYCQQTNEDPYTFGQGTKVEIK

SEQ ID NO: 7 - protein, 15 aa, Synthetic construct
RAGESVDIFGVGFLH

SEQ ID NO: 8 - protein, 7 aa, Synthetic construct
RASNLES

SEQ ID NO: 9 - protein, 9 aa, Synthetic construct
QQTNEDPYT

SEQ ID NO: 10 - protein, 114 aa, Synthetic construct
EVQLVESGGGLVQPGGSLRLSCAASGFNIKDTYMHWVRQAPGKGLEWVARIDPANGNSKYADSVKGRFTISADTSKNTAYLQMNSLRAEDTAVYYCAPFGYYVSDYAMAYWGQG

SEQ ID NO: 11 - protein, 5 aa, Synthetic construct
DTYMH

SEQ ID NO: 12 - protein, 17 aa, Synthetic construct
RIDPANGNSKYADSVKG

SEQ ID NO: 13 - protein, 12 aa, Synthetic construct
FGYYVSDYAMAY

SEQ ID NO: 14 - protein, 114 aa, Synthetic construct
EVQLVESGGGLVQPGGSLRLSCAASGFNIKDTYMHWVRQAPGKGLEWVARIDPANGNSKYVPKFQGRATISADTSKNTAYLQMNSLRAEDTAVYYCAPFGYYVSDYAMAYWGQG

SEQ ID NO: 15 - protein, 17 aa, Synthetic construct
RIDPANGNSKYVPKFQG

SEQ ID NO: 16 - protein, 4 aa, Synthetic construct
GGGS

SEQ ID NO: 17 - protein, 5 aa, Synthetic construct
GGGGS

SEQ ID NO: 18 - protein, 40 aa, Homo sapiens
SKRSRLLHSDYMNMTPRRPGPTRKHYQPYAPPRDFAAYRS

SEQ ID NO: 19 - protein, 21 aa, Homo sapiens
LCYLLDGILFIYGVILTALFL

SEQ ID NO: 20 - protein, 27 aa, Homo sapiens
FWVLVVVGGVLACYSLLVTVAFIIFWV

SEQ ID NO: 21 - protein, 28 aa, Homo sapiens
MFWVLVVVGGVLACYSLLVTVAFIIFWV

SEQ ID NO: 22 - protein, 22 aa, Homo sapiens
MALIVLGGVAGLLLFIGLGIFF

SEQ ID NO: 23 - protein, 21 aa, Homo sapiens
IYIWAPLAGTCGVLLLSLVIT

SEQ ID NO: 24 - protein, 23 aa, Homo sapiens
IYIWAPLAGTCGVLLLSLVITLY

SEQ ID NO: 25 - protein, 24 aa, Homo sapiens
IYIWAPLAGTCGVLLLSLVITLYC

SEQ ID NO: 26 - protein, 27 aa, Homo sapiens
IISFFLALTSTALLFLLFFLTLRFSVV

SEQ ID NO: 27 - protein, 21 aa, Homo sapiens
PFFFCCFIAVAMGIRFIIMVA

SEQ ID NO: 28 - protein, 10 aa, Synthetic construct
GGGSSGGGSG

SEQ ID NO: 29 - protein, 12 aa, Synthetic construct
ESKYGPPCPPCP

SEQ ID NO: 30 - protein, 12 aa, Homo sapiens
ESKYGPPCPSCP

SEQ ID NO: 31 - protein, 22 aa, Synthetic construct
ESKYGPPCPPCPGGGSSGGGSG

SEQ ID NO: 32 - protein, 39 aa, Homo sapiens
IEVMYPPPYLDNEKSNGTIIHVKGKHLCPSPLFPGPSKP

SEQ ID NO: 33 - protein, 48 aa, Homo sapiens
AKPTTTPAPRPPTPAPTIASQPLSLRPEACRPAAGGAVHTRGLDFACD

SEQ ID NO: 34 - protein, 45 aa, Homo sapiens
TTTPAPRPPTPAPTIASQPLSLRPEACRPAAGGAVHTRGLDFACD

SEQ ID NO: 35 - protein, 129 aa, Synthetic construct
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 229 aa, Synthetic construct
ESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 229 aa, Synthetic construct
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 112 aa, Synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 41 - protein, 112 aa, Synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLYQGLSTATKDTYDALHMQALPPR

SEQ ID NO: 42 - protein, 112 aa, Synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLFQGLSTATKDTFDALHMQALPPR

SEQ ID NO: 43 - protein, 112 aa, Synthetic construct
RVKFSRSADAPAYQQGQNQLYNELNLGRREEYDVLDKRRGRDPEMGGKPRRKNPQEGLYNELQKDKMAEAFSEIGMKGERRRGKGHDGLFQGLSTATKDTFDALHMQALPPR

SEQ ID NO: 44 - protein, 112 aa, Synthetic construct
RVKFSRSADAPAYQQGQNQLYNELNLGRREEYDVLDKRRGRDPEMGGKPRRKNPQEGLFNELQKDKMAEAYSEIGMKGERRRGKGHDGLFQGLSTATKDTFDALHMQALPPR

SEQ ID NO: 45 - protein, 112 aa, Synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLYQGLSTATKDTFDALHMQALPPR

SEQ ID NO: 46 - protein, 112 aa, Synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTYDALHMQALPPR

SEQ ID NO: 47 - protein, 41 aa, Homo sapiens
RSKRSRLLHSDYMNMTPRRPGPTRKHYQPYAPPRDFAAYRS

SEQ ID NO: 48 - protein, 41 aa, Synthetic construct
RSKRSRGGHSDYMNMTPRRPGPTRKHYQPYAPPRDFAAYRS

SEQ ID NO: 49 - protein, 42 aa, Homo sapiens
KRGRKKLLYIFKQPFMRPVQTTQEEDGCSCRFPEEEEGGCEL

SEQ ID NO: 50 - protein, 42 aa, Homo sapiens
ALYLLRRDQRLPPDAHKPPGGGSFRTPIQEEQADAHSTLAKI

SEQ ID NO: 51 - protein, 120 aa, Homo sapiens
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 52 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 53 - protein, 450 aa, Synthetic construct
EVQLVESGGGLVQPGGSLRLSCAASGFNIKDTYMHWVRQAPGKGLEWVARIDPANGNSKYADSVKGRFTISADTSKNTAYLQMNSLRAEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSATATPG

SEQ ID NO: 54 - protein, 218 aa, Synthetic construct
DIQLTQSPSSLSASVGDRVTITCRAGESVDIFGVGFLHWYQQKPGKAPKLLIYRASNLESGVPSRFSGSGSRTDFTLTISSLQPEDFATYYCQQTNEDPYTFGQGTKVEIKRTVAAPSVFIFPPSDEQLKSGAASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 153 aa, Homo sapiens [sequence omitted]

SEQ ID NO: 56 - protein, 133 aa, Homo sapiens [sequence omitted]

SEQ ID NO: 57 - protein, 24 aa, Synthetic construct
LEGGGEGRGSLLTCGDVEENPGPR

SEQ ID NO: 58 - protein, 354 aa, Homo sapiens
LVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 59 - protein, 323 aa, Homo sapiens
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 60 - protein, 583 aa, Synthetic construct
EVQLVESGGGLVQPGGSLRLSCAASGFNIKDTYMHWVRQAPGKGLEWVARIDPANGNSKYADSVKGRFTISADTSKNTAYLQMNSLRAEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSATATPGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 61 - protein, 131 aa, Synthetic construct
METDTLLLWVLLLWVPGSTGDIQLTQSPSSLSASVGDRVTITCRAGESVDIFGVGFLHWYQQKPGKAPKLLIYRASNLESGVPSRFSGSGSRTDFTLTISSLQPEDFATYYCQQTNEDPYTFGQGTKVEIK

SEQ ID NO: 62 - protein, 107 aa, Synthetic construct
RTVAAPSVFIFPPSDEQLKSGAASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 121 aa, Synthetic construct
EVQLVESGGGLVQPGGSLRLSCAASGFNIKDTYMHWVRQAPGKGLEWVARIDPANGNSKYADSVKGRFTISADTSKNTAYLQMNSLRAEDTAVYYCAPFGYYVSDYAMAYWGQGTLVTVSS

SEQ ID NO: 64 - protein, 108 aa, Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 65 - protein, 499 aa, Synthetic construct
METDTLLLWVLLLWVPGSTGDIQLTQSPSSLSASVGDRVTITCRAGESVDIFGVGFLHWYQQKPGKAPKLLIYRASNLESGVPSRFSGSGSRTDFTLTISSLQPEDFATYYCQQTNEDPYTFGQGTKVEIKRTVAAPSVFIFPPSDEQLKSGAASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGEVQLVESGGGLVQPGGSLRLSCAASGFNIKDTYMHWVRQAPGKGLEWVARIDPANGNSKYADSVKGRFTISADTSKNTAYLQMNSLRAEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 66 - protein, 812 aa, Synthetic construct
METDTLLLWVLLLWVPGSTGDIQLTQSPSSLSASVGDRVTITCRAGESVDIFGVGFLHWYQQKPGKAPKLLIYRASNLESGVPSRFSGSGSRTDFTLTISSLQPEDFATYYCQQTNEDPYTFGQGTKVEIKRTVAAPSVFIFPPSDEQLKSGAASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGEVQLVESGGGLVQPGGSLRLSCAASGFNIKDTYMHWVRQAPGKGLEWVARIDPANGNSKYADSVKGRFTISADTSKNTAYLQMNSLRAEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 919 aa, Synthetic construct
METDTLLLWVLLLWVPGSTGDIQLTQSPSSLSASVGDRVTITCRAGESVDIFGVGFLHWYQQKPGKAPKLLIYRASNLESGVPSRFSGSGSRTDFTLTISSLQPEDFATYYCQQTNEDPYTFGQGTKVEIKRTVAAPSVFIFPPSDEQLKSGAASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGEVQLVESGGGLVQPGGSLRLSCAASGFNIKDTYMHWVRQAPGKGLEWVARIDPANGNSKYADSVKGRFTISADTSKNTAYLQMNSLRAEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 1159 aa, Synthetic construct
METDTLLLWVLLLWVPGSTGDIQLTQSPSSLSASVGDRVTITCRAGESVDIFGVGFLHWYQQKPGKAPKLLIYRASNLESGVPSRFSGSGSRTDFTLTISSLQPEDFATYYCQQTNEDPYTFGQGTKVEIKRTVAAPSVFIFPPSDEQLKSGAASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGEVQLVESGGGLVQPGGSLRLSCAASGFNIKDTYMHWVRQAPGKGLEWVARIDPANGNSKYADSVKGRFTISADTSKNTAYLQMNSLRAEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 69 - protein, 1266 aa, Synthetic construct
METDTLLLWVLLLWVPGSTGDIQLTQSPSSLSASVGDRVTITCRAGESVDIFGVGFLHWYQQKPGKAPKLLIYRASNLESGVPSRFSGSGSRTDFTLTISSLQPEDFATYYCQQTNEDPYTFGQGTKVEIKRTVAAPSVFIFPPSDEQLKSGAASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGEVQLVESGGGLVQPGGSLRLSCAASGFNIKDTYMHWVRQAPGKGLEWVARIDPANGNSKYADSVKGRFTISADTSKNTAYLQMNSLRAEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 70 - protein, 848 aa, Synthetic construct
MLLLVTSLLLCELPHPAFLLIPDIVLTQSPASLAVSLGQRATMSCRAGESVDIFGVGFLHWYQQKPGQPPKLLIYRASNLESGIPVRFSGTGSRTDFTLIIDPVEADDVATYYCQQTNEDPYTFGGGTKLEIKGSTSGGGSGGGSGGGGSSEVQLQQSGAELVEPGASVKLSCTASGFNIKDTYMHWVKQRPEQGLEWIGRIDPANGNSKYVPKFQGKATITADTSSNTAYLQLTSLTSEDTAVYYCAPFGYYVSDYAMAYWGQGTSVTVSSTTTKPVLRTPSPVHPTGTSQPQRPEDCRPRGSVKGTGLDFACDIYMFWALVVVAGVLFCYGLLVTVALCVIWTNSRRNRLLQSDYMNMTPRRPGLTRKPYQPYAPARDFAAYRPGGGRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRLEGGGEGRGSLLTCGDVEENPGPTRMPSPLPVSFLLFLTLVGGRPQKSLLVEVEEGGNVVLPCLPDSSPVSSEKLAWYRGNQSTPFLELSPGSPGLGLHVGSLGILLVIVNVSDHMGGFYLCQKRPPFKDIWQPAWTVNVEDSGEMFRWNASDVRDLDCDLRNRSSGSHRSTSGSQLYVWAKDHPKVWGTKPVCAPRGSSLNQSLINQDLTVAPGSTLWLSCGVPPVPVAKGSISWTHVHPRRPNVSLLSLSLGGEHPVREMWVWGSLLLLPQATALDEGTYYCLRGNLTIERHVKVIARSAVWLWLLRTGGWIVPVVTLVYVIFCMVSLVAFLYCQRAFILRRKR

(19) United States
(12) Patent Application Publication
(10) Pub. No.: US 2023/0190799 A1
(43) Pub. Date: Jun. 22, 2023

(54) CHIMERIC ANTIGEN RECEPTOR T CELLS TARGETING CEA AND ANTI-CEA-IL2 IMMUNOCYTOKINES FOR CANCER THERAPY

(71) Applicant: City of Hope, Duarte, CA (US)

(72) Inventors: John E. Shively, Arcadia, CA (US); Maciej Kujawski, Glendora, CA (US); Seung E. Cha, Claremont, CA (US); Paul Yazaki, Glendale, CA (US)

(21) Appl. No.: 17/870,024

(22) Filed: Jul. 21, 2022

Related U.S. Application Data
(60) Provisional application No. 63/224,061, filed on Jul. 21, 2021.

(51) Int. Cl. A61K 35/17; C07K 16/30; A61K 31/664; A61P 35/00; A61P 1/00; A61K 38/20; A61K 38/17; A61K 39/395

(52) U.S. Cl. CPC A61K 35/17; C07K 16/3007; A61K 31/664; A61P 35/00; A61P 1/00; A61K 38/2013; A61K 38/1774; A61K 39/39558; C07K 2317/622; C07K 2317/55; A61K 2039/545

(57) ABSTRACT

Aspects of the present disclosure provide methods for treating a subject having a carcinoembryonic antigen (CEA)-positive tumor using a conditioning regimen (lymphodepleting treatment), which comprises administering one or more doses of a lymphodepleting agent to a subject, and a treatment regimen, which comprises administrating one or more doses of the anti-CEA CAR T cells and/or the ICK proteins to the subject.

### CLAIM OF PRIORITY

This application claims priority under 35 U.S.C. § 119(e) to U.S. Provisional Application No. 63/224,061, filed on Jul. 21, 2021, the entire contents of which are hereby incorporated by reference.

### SEQUENCE LISTING

This application contains a Sequence Listing that has been submitted electronically as an XML file named 40056-0074001_SL_ST26.xml. The XML file, created on Jan. 4, 2023, is 73,185 bytes in size. The material in the XML file is hereby incorporated by reference in its entirety.

### TECHNICAL FIELD

This disclosure relates to treating cancer using anti-CEA CAR T cell therapy and anti-CEA-IL2 immunocytokines (ICK).

### BACKGROUND

Chimeric antigen receptor (CAR) engineered T cells have energized the field of cancer immunotherapy with their proven ability to treat hematological malignancies, yet the success of CAR T cells against solid tumors has been limited. Several factors can be attributed to the lack of success of CAR T cell therapy against solid tumors including the antigen heterogeneity of solid tumors, the difficulty trafficking CAR T cells to solid tumors, and the effectiveness of inhibitory signals produced by the solid tumor against the CAR T cell therapy. Thus, there is a need for CAR T cell therapies that are effective against solid tumors.

### SUMMARY

The present disclosure is based, at least in part, on the surprising discovery that anti-CEA CAR T cell therapy in combination with administration of an anti-CEA-IL2 immunocytokine (anti-CEA antibody-IL-2 fusion protein; “anti-CEA-IL2 ICK”) reduced tumor burden in subcutaneous breast and colon cancer xenograft models. It was also demonstrated that treatment with the anti-CEA CAR T cells in combination with multiple administrations of the anti-CEA-IL2 ICK provided long-term in vivo efficacy that prevented tumor growth after re-exposure to tumor cells. Significant reductions in tumor burden were also observed after treatment with cyclophosphamide (CY) in combination with multiple administrations of the anti-CEA-IL-2 ICK.

Accordingly, aspects of the present disclosure provide methods for treating a subject having a cancer characterized by growth of tumor cells expressing carcinoembryonic antigen (CEA) comprising administering to the subject a population of T cells expressing a chimeric antigen receptor (CAR) that binds CEA and an anti-CEA-IL-2 immunocytokine (ICK), wherein the CAR that binds CEA comprises a single-chain variable fragment (scFv) or an antigen-binding fragment (Fab) that binds CEA, a spacer domain, a transmembrane domain, a co-stimulatory domain, and a CD3ζ cytoplasmic signaling domain, and wherein the anti-CEA-IL-2 ICK comprises a heavy chain variable domain (V_{H}), a light chain variable domain (V_{L}), and IL-2.

In some embodiments, the population of T cells is administered 1 to 3 days prior to administering the anti-CEA-IL-2 ICK.

In some embodiments, methods further comprise administering at least one additional dose of the anti-CEA-IL-2 ICK. In some embodiments, the at least one additional dose of the anti-CEA-IL-2 ICK comprises 3 to 6 doses, each of which is administered 1 to 5 days after the prior dose.

In some embodiments, methods further comprise administering to the subject a lymphodepleting agent. In some embodiments, the lymphodepleting agent is administered 1 to 3 days prior to administering the population of T cells. In some embodiments, the lymphodepleting agent is cyclophosphoamide (CY).

In some embodiments, methods further comprise treating the subject with an additional anti-cancer therapy. In some embodiments, the additional anti-cancer therapy is stereotactic radiation therapy (SRT).

In some embodiments, the scFv or the Fab of the CAR comprises a light chain complementary determining region 1 (LC CDR1) of SEQ ID NO: 7, a light chain complementary determining region 2 (LC CDR2) of SEQ ID NO: 8, and a light chain complementary determining region 3 (LC CDR3) of SEQ ID NO: 9, and a heavy chain complementary determining region 1 (HC CDR1) of SEQ ID NO: 11, a heavy chain complementary determining region 2 (HC CDR2) of SEQ ID NO: 12 or SEQ ID NO: 15, and a heavy chain complementary determining region 3 (HC CDR3) of SEQ ID NO: 13.

In some embodiments, the scFv of the CAR comprises a V_{L} comprising SEQ ID NO: 6, and a V_{H} comprising SEQ ID NO: 10 or SEQ ID NO: 14. In some embodiments, the scFv of the CAR comprises SEQ ID NO: 5.

In some embodiments, the Fab comprises a V_{L} comprising SEQ ID NO: 61, a light chain constant region (C_{L}) comprising SEQ ID NO: 62, a V_{H} comprising SEQ ID NO: 63, and a heavy chain constant region 1 (C_{H}1) comprising SEQ ID NO: 64. In some embodiments, the Fab comprises SEQ ID NO: 65.

In some embodiments, the spacer domain comprises an a3 spacer domain, a linker domain, an IgG4 hinge or variant thereof, a CD28 hinge or a variant thereof, a CD8 hinge, or a combination thereof. In some embodiments, the spacer domain comprises any one of SEQ ID NOs: 28-38.

In some embodiments, the transmembrane domain comprises a CD3ζ transmembrane domain, a CD8 transmembrane domain, a CD28 transmembrane domain, a CD4 transmembrane domain, a 41BB transmembrane domain, a NKG2D transmembrane domain, or a combination thereof. In some embodiments, the transmembrane domain comprises any one of SEQ ID NOs: 18-27.

In some embodiments, the co-stimulatory domain is selected from the group consisting of a CD3ζ co-stimulatory domain or variant thereof, a CD28 co-stimulatory domain or variant thereof, a 41BB co-stimulatory domain, an OX40 co-stimulatory domain, a 2B4 co-stimulatory domain, a CTLA-4 co-stimulatory domain, or a combination thereof. In some embodiments, the co-stimulatory domain comprises any one of SEQ ID NOs: 39-52.

In some embodiments, the CD3ζ co-stimulatory domain comprises SEQ ID NO: 39.

In some embodiments, the CAR comprises SEQ ID NO: 66. In some embodiments, the CAR comprises SEQ ID NO: 67.

In some embodiments, the V_{L} of the anti-CEA-IL-2 ICK comprises a LC CDR1 of SEQ ID NO: 7, a LC CDR2 of SEQ ID NO: 8, and a LC CDR3 of SEQ ID NO: 9, and the V_{H} of the anti-CEA-IL-2 ICK comprises a HC CDR1 of SEQ ID NO: 11, a HC CDR2 of SEQ ID NO: 12 or SEQ ID NO: 15, and a HC CDR3 of SEQ ID NO: 13. In some embodiments, the V_{L} of the anti-CEA-IL-2 ICK comprises SEQ ID NO: 6, and the V_{H} of the anti-CEA-IL-2 ICK comprises SEQ ID NO: 10 or SEQ ID NO: 14.

In some embodiments, the anti-CEA-IL-2 ICK comprises a heavy chain comprising SEQ ID NO: 53 and a light chain comprising SEQ ID NO: 54. In some embodiments, the anti-CEA-IL-2 ICK comprises IL-2 comprising SEQ ID NO: 55 or SEQ ID NO: 56.

In some embodiments, the V_{H} and the V_{L} of the CAR are identical to the V_{H} and V_{L} of the anti-CEA-IL-2 ICK.

In some embodiments, the CAR and the anti-CEA-IL-2 ICK target the A3 domain of

CEA.

In some embodiments, the cancer is a gastrointestinal cancer, a breast cancer, or a lung cancer. In some embodiments, the gastrointestinal cancer is selected from the group consisting of a colon cancer, a gastric cancer, a rectal cancer, and a pancreatic cancer.

In some embodiments, the subject is a human patient.

In some embodiments, methods further comprise administering at least one additional dose of the population of T cells expressing the CAR.

In some embodiments, the population of T cells and the ICK are administered simultaneously or sequentially.

Aspects of the present disclosure provide methods for treating a subject having a cancer characterized by growth of tumor cells expressing carcinoembryonic antigen (CEA) comprising administering to the subject a lymphodepleting agent and multiple doses of an anti-CEA-IL-2 immunocytokine (ICK), wherein the anti-CEA-IL-2 ICK comprises a heavy chain variable domain (V_{H}), a light chain variable domain (V_{L}), and IL-2, and wherein a first dose of the multiple doses of the anti-CEA-IL-2 ICK is administered 1 to 3 days after the administration of the lymphodepleting agent.

In some embodiments, administering multiple doses of the anti-CEA IL-2 ICK comprises administering 4 doses of the anti-CEA IL-2 ICK, each of which is administered 1 to 4 days after the previous dose.

In some embodiments, the lymphodepleting agent is cyclophosphoamide (CY).

In some embodiments, methods further comprise administering to the subject a population of T cells expressing a chimeric antigen receptor (CAR) that binds CEA, wherein the CAR that binds CEA comprises a single-chain variable fragment (scFv) or an antigen-binding fragment (Fab) that binds CEA, a spacer domain, a transmembrane domain, a co-stimulatory domain, and a CD3ζ cytoplasmic signaling domain.

Aspects of the present disclosure provide a population of cells comprising T cells expressing a chimeric antigen receptor (CAR) that binds CEA, wherein the CAR that binds CEA comprises a single-chain variable fragment (scFv) or an antigen-binding fragment (Fab) that binds CEA, a spacer domain, a transmembrane domain, a co-stimulatory domain, and a CD3 ζ cytoplasmic signaling domain.

In some embodiments, the scFv or the Fab of the CAR comprises a light chain complementary determining region 1 (LC CDR1) of SEQ ID NO: 7, a light chain complementary determining region 2 (LC CDR2) of SEQ ID NO: 8, and a light chain complementary determining region 3 (LC CDR3) of SEQ ID NO: 9, and a heavy chain complementary determining region 1 (HC CDR1) of SEQ ID NO: 11, a heavy chain complementary determining region 2 (HC CDR2) of SEQ ID NO: 12 or SEQ ID NO: 15, and a heavy chain complementary determining region 3 (HC CDR3) of SEQ ID NO: 13.

In some embodiments, the scFv of the CAR comprises a V_{L} comprising SEQ ID NO: 6, and a V_{H} comprising SEQ ID NO: 10 or SEQ ID NO: 14. In some embodiments, the scFv of the CAR comprises SEQ ID NO: 5.

In some embodiments, the Fab CAR comprises a V_{L} comprising SEQ ID NO: 61, a light chain constant region (CL) comprising SEQ ID NO: 62, a V_{H} comprising SEQ ID NO: 63, and a heavy chain constant region 1 (C_{H}1) comprising SEQ ID NO: 64. In some embodiments, the Fab CAR comprises SEQ ID NO: 65.

In some embodiments, the spacer domain comprises an a3 spacer domain, a linker domain, an IgG4 hinge or variant thereof, a CD28 hinge or a variant thereof, a CD8 hinge, or a combination thereof. In some embodiments, the spacer domain comprises any one of SEQ ID NOs: 28-38.

In some embodiments, the transmembrane domain comprises a CD3ζ transmembrane domain, a CD8 transmembrane domain, a CD28 transmembrane domain, a CD4 transmembrane domain, a 41BB transmembrane domain, a NKG2D transmembrane domain, or a combination thereof. In some embodiments, the transmembrane domain comprises any one of SEQ ID NOs: 18-27.

In some embodiments, the co-stimulatory domain is selected from the group consisting of a CD3ζ co-stimulatory domain or variant thereof, a CD28 co-stimulatory domain or variant thereof, a 41BB co-stimulatory domain, an OX40 co-stimulatory domain, a 2B4 co-stimulatory domain, a CTLA-4 co-stimulatory domain, or a combination thereof. In some embodiments, the co-stimulatory domain comprises any one of SEQ ID NOs: 39-52. In some embodiments, wherein the CD3ζ co-stimulatory domain comprises SEQ ID NO: 39.

In some embodiments, the population of cells further expresses an anti-CEA-IL-2 immunocytokine (ICK), wherein the anti-CEA-IL-2 ICK comprises a heavy chain variable domain (V_{H}), a light chain variable domain (V_{L}), and IL-2.

In some embodiments, the V_{L} of the anti-CEA-IL-2 ICK comprises a LC CDR1 of SEQ ID NO: 7, a LC CDR2 of SEQ ID NO: 8, and a LC CDR3 of SEQ ID NO: 9, and the V_{H} of the anti-CEA-IL-2 ICK comprises a HC CDR1 of SEQ ID NO: 11, a HC CDR2 of SEQ ID NO: 12 or SEQ ID NO: 15, and a HC CDR3 of SEQ ID NO: 13. In some embodiments, the V_{L} of the anti-CEA-IL-2 ICK comprises SEQ ID NO: 6, and the V_{H} of the anti-CEA-IL-2 ICK comprises SEQ ID NO: 10 or SEQ ID NO: 14.

In some embodiments, the anti-CEA-IL-2 ICK comprises a heavy chain comprising SEQ ID NO: 53 and a light chain comprising SEQ ID NO: 54. In some embodiments, the anti-CEA-IL-2 ICK comprises IL-2 comprising SEQ ID NO: 55 or SEQ ID NO: 56.

In some embodiments, the V_{H} and the V_{L} of the CAR are identical to the V_{H} and V_{L} of the anti-CEA-IL-2 ICK.

In some embodiments, the CAR and the anti-CEA-IL-2 ICK target the A3 domain of CEA. In some embodiments, the CAR comprises SEQ ID NO: 66. In some embodiments, the CAR comprises SEQ ID NO: 67.

### BRIEF DESCRIPTION OF THE DRAWINGS

FIGS. 1A-1G include data showing that anti-CEA CAR T cells specifically target CEA-positive mouse adenocarcinoma cells in vitro. FIG. 1A includes a diagram of the retroviral expression cassettes with a mock control containing truncated murine CD19 (mCD19t) and the anti-CEA CAR cassette containing the scFv from murine anti-CEA antibody T84.66, mCD28 transmembrane domain, mCD28 cytoplasmic domain, and mCD3ζ cytoplasmic domain with mCD19t separated by T2A ribosomal skip sequence. FIG. 1B includes data showing transduction efficiency evaluated by flow cytometry for CD19t expression on transduced T cells. FIG. 1C includes data showing CEA and GFP expression on mouse colon (MC38/GFP or MC38/CEA/GFP) and breast (E0771/GFP or E0771/CEA/GFP) adenocarcinoma cells evaluated by flow cytometry. Quantification of cell cytotoxicity (squares for CAR; circles for Mock) of either (FIGS. 1D-1E) mouse colon or (FIGS. 1F-1G) mouse breast adenocarcinoma cells and INFγ production by mock transfected (rectangle) or anti-CEA CAR T cells (straight line) following 24 hour co-culture at an increasing effector:target (E:T) ratios. (n=6 per group). ****p<0.0001; ***p<0.001; **p<0.01; *p<0.05.

FIGS. 2A-2E include data showing improved effect of cyclophosphamide plus anti-CEA CAR T cells on s.c. MC38/CEA and orthotopic E0771/CEA tumor growth inhibition in immunocompetent mice transgenic for human CEA (CEATg) mouse model (Clarke et al. Cancer Research, 1998 Apr. 1, 58(7) 1469-77; Xu et al. Cancer research 2000, Aug 15; 60(16) 4475-84). FIG. 2A includes a graph of effects of cyclophosphamide (CY) on lymphocyte populations of tumor-bearing CEATg mice. First bar for before and second bar for after CY. Tumor volume (mm³) and survival of (FIGS. 2B-2C) s.c. MC38/CEA (1×10⁶) or (FIGS. 2D-2E) orthotopic E0771/CEA (1×10⁵) tumor-bearing CEATg mice treated after 12 or 7 days of tumor implantation, respectively, with i.p. injection of CY (100 mg/kg mouse weight) followed by i.v. injection of either 5×10⁶ mock transduced or anti-CEA CAR T cells on the next day. For the control group, tumor-bearing mice were i.p. injected with CY alone.

Mice were euthanized when tumor volumes reached 1500 mm³. Anti-CEA CAR T cell therapy was statistically significant measured at 30 days for MC38/CEA tumors (p<0.0001) and at 21 days for E0771/CEA tumors (p<0.0001) post tumor implantation. Efficacy of single anti-CEA CART cell therapy on the survival of s.c. MC38/CEA tumor-bearing mice (p<0.05) and orthotopic E0771/CEA tumor-bearing mice (p<0.05). FIGS. 2B-2E: Light grey for CY only (n=4-7); black for CY+Mock (n=6-7); dark grey for CY+CAR (n=7). ****p<0.0001; *p<0.05.

FIG. 3 includes images of tissue sections showing CEA expression on s.c. MC38/CEA tumors in CEATg mice. After s.c. MC38/CEA tumor has grown to 1500 mm³ the termination tumor size in CEATg mice, tumors were collected and analyzed on immunohistochemistry (IHC) for human CEA. The tumor-bearing mice were treated at day 12 after tumor implantation with a combination of CY, mock transduced T cells, and/or anti-CEA CAR T cells.

FIGS. 4A-4D include data showing therapeutic efficacy of cyclophosphamide plus repeated anti-CEA CAR T cell therapy on s.c. MC38/CEA tumor in CEATg mice. FIG. 4A includes a schematic depiction of the treatment regimen in which subcutaneous MC38/CEA tumor-bearing CEATg mice were treated on day 15 with i.p. injection of CY (100 mg/kg) followed by three i.v. injections of either 5×10⁶ Mock or anti-CEA CART cells on days 16, 23, and 40. FIG. 4B includes a graph of tumor volume for each group, which was measured until they reached 1500 mm³. Anti-CEA CAR T cell therapy was statistically significant up to 23 days post tumor implantation (p<0.001). FIG. 4C includes a graph of efficacy of CY plus repeated anti-CEA CAR T cell therapy on the survival of s.c. MC38/CEA tumor-bearing mice (p<0.01). FIG. 4D includes a graph of average weight of tumor-bearing CEATg mice separated by sex in each treatment group (upper graphs are males). FIGS. 4B-4D: Black circles and squares for CY+Mock; grey triangles for CY+CAR. (n=9 per group).

FIGS. 5A-5D includes graphs showing effects of anti-CEA-IL2 immunocytokine (ICK) on anti-CEA CAR T cell activity in vitro. Quantification of cell cytotoxicity of either (FIG. 5A) MC38/CEA/GFP or (FIG. 5B) MC38/GFP cells and (FIGS. 5C-5D) IFNγ production by mock or anti-CEA CAR T cells following 24 hour co-culture with ICK (12 ng/mL) at an increasing effector:target (E:T) ratios. (n=6 per group). FIGS. 5A-5D: Black circles for Mock; black squares for CAR; grey upward pointing triangle for Mock+ICK; black downward pointing triangle for CAR+ICK. ****p<0.0001; ***p<0.001; *p<0.05.

FIGS. 6A-6I includes data showing therapeutic efficacy of anti-CEA-IL2 immunocytokine (ICK) on CY plus anti-CEA CAR T cells on s.c. MC38/CEA and orthotopic E0771/CEA tumors in CEATg mice. FIG. 6A includes a schematic depiction of the treatment regimen in which subcutaneous MC38/CEA (1×10⁶) tumor-bearing CEATg mice were treated on day 11 post tumor implantation with i.p. injection of CY (100 mg/kg) followed by i.v. injection of either 5×10⁶ mock or anti-CEA CART cells on day 12 and i.p. injection of anti-CEA-IL-2 ICK (25 μg/mouse) on day 13. FIG. 6B includes a graph of mean tumor volume. Due to tumor ulceration, most mice had to be euthanized before reaching 1500 mm³. Combined therapy of CY, anti-CEA CAR T cells, and ICK was statistically significant up to 29 days post tumor implantation (p<0.001). Circles for CY only (n=5); squares for CY+ICK (n=6); upward pointing triangles for CY+CAR (n=8); downward pointing triangles for CY+CAR+ICK (n=8). FIG. 6C includes a schematic depiction of the treatment regimen in which orthotopic E0771/CEA (1×10⁵) tumor-bearing CEATg mice were treated on day 7 post tumor implantation with i.p. injection of CY (100 mg/kg) followed by i.v. injection of either 5×10⁶ mock or anti-CEA CART cells on day 8 and i.p. injection of anti-CEA-IL-2 ICK (25 μg/mouse) on day 9 and 12. FIG. 6D includes a graph of mean tumor volume. Combined therapy of CY, anti-CEA CAR T cells, and ICK was statistically significant up to 25 days post tumor implantation (p<0.001). Triangles for CY+ICK; circles for CY+Mock+ICK; squares for CY+CAR+ICK. (n=6 per group). FIG. 6E includes a graph of average weight of tumor-bearing mice in each treatment group. Circles for CY+ICK; squares for CY+Mock+ICK; triangles for CY+CAR+ICK. (n=6 per group). FIG. 6F includes a schematic depiction of the treatment regimen in which subcutaneous MC38/CEA tumor-bearing CEATg mice were treated on day 13 post tumor implantation with i.p. injection of CY followed by i.v. injection of either mock or anti-CEA CAR T cells on day 14 and i.p. injections of anti-CEA-IL-2 ICK (25 μg/mouse) on day 15, 18, 21, and 24. FIG. 6G includes a graph of tumor volume (mm³) measured until it reached 1500 mm³. Combined therapy of CY, anti-CEA CAR T cells, and ICK was statistically significant up to 48 days post tumor implantation (p<0.0001). FIG. 6H includes a graph of tumor volume (mm³) after tumor re-challenge. After 45 days of being tumor-free, MC38/CEA (1×10⁶) cells were i.p. injected for a tumor re-challenge. FIG. 61 includes a graph of average weight of tumor-bearing mice in each treatment group. FIGS. 6G-6I: Circles for CY only (n=4); triangles for CY+ICK (n=4); upward pointing triangles for CY+CAR (n=6); downward pointing triangles for CY+CAR+ICK (n=6).

FIGS. 7A-7D include data showing therapeutic efficacy of anti-CEA CAR T cells on s.c. MC38/CEA tumors in CEATg mice. FIG. 7A includes a graph of tumor volume (mm³) of s.c. MC38/CEA tumor-bearing CEATg mice treated with i.v. injection of either 5×10⁶ mock transduced or anti-CEA CAR T cells after 13 days of tumor growth. The tumor volume was measured until it reached 1500 mm³. Tumor growth inhibition by CAR T cell therapy was statistically significant compared to mock transduced controls up to 24 days post tumor implantation (p<0.05). FIG. 7B includes a graph of tumor volume showing that one of 5 to MC38/CEA tumor-bearing mice showed complete regression after single anti-CEA CAR T cell therapy. After 27 days of being tumor-free, the tumor came back and grew to 1500 mm³ within 25 days. FIG. 7C includes a graph showing efficacy of single anti-CEA CAR T cell therapy on the survival of s.c. MC38/CEA tumor-bearing CEATg mice based on 4 folds increase of initial tumor size (p<0.05). FIG. 7D includes a graph of average weight of mice treated with either mock or anti-CEA CAR T cell therapy. FIGS. 7A-7D: Black for mock (n=6); grey for CAR (n=5).

FIGS. 8A-8B include data showing effect of cyclophosphamide on CEA-negative and CEA-positive mouse colon carcinoma (MC38) tumors in CEATg mice. MC38 or MC38/CEA mouse colon adenocarcinoma cells (1×10⁶ cells) were subcutaneously injected into CEATg mice. Ten days after colon adenocarcinoma cells injection, cyclophosphamide (CY; 100 mg/kg mouse weight) was injected into CEATg mice for lymphodepletion. MC38 (FIG. 8A) and MC38/CEA (FIG. 8B) colon adenocarcinoma tumor volumes were measured with electronic caliper for 48 and 32 days, respectively, after the lymphodepletion via CY treatment.

FIGS. 9A-9B include data showing that no severe toxicity was detected after cyclophosphamide plus anti-CEA CAR T cell treatments on s.c. MC38/CEA or orthotopic E0771/CEA tumor-bearing CEATg mice. Average weight of s.c. MC38/CEA (FIG. 9A) or orthotopic E0771/CEA (FIG. 9B) tumor-bearing CEATg mice treated after 12 or 7 days of tumor implantation, respectively, with i.p. injection of CY (100 mg/kg mouse weight) followed by i.v. injection of either 5×10⁶ mock transduced or anti-CEA CART cells on the next day. For the control group, tumor-bearing mice were i.p. injected with CY alone. FIG. 9A: Upward pointing triangles for CY only (n=4-7); circles for CY+Mock (n=6-7); squares for CY+CAR (n=7). FIG. 9B: Circles for CY only (n=4-7); squares for CY+Mock (n=6-7); upward pointing triangles for CY+CAR (n=7).

FIG. 10 include images of tissue sections showing CEA expression on CEA-positive tissues in CEATg mice. CEA-positive tissues such as lung, stomach, intestine, and colon were collected from tumor-bearing mice three days after being treated with i.v. injection of T cell (5×10⁶) therapy. The tissues were analyzed on immunohistochemistry (IHC) for human CEA.

FIGS. 11A-11B include images of tissue sections showing immune cell infiltrates into CEA-positive tissues in CEATg mice. CEA-positive tissues such as lung, stomach, intestine, and colon were collected from tumor-bearing mice treated with i.p. injection of CY (100 mg/kg) followed by i.v. injection of CAR T cell therapy. The tissues were analyzed on to immunohistochemistry (IHC) for mouse CD3⁺ T cells three days after T cell therapy (FIG. 11A) and at the termination timepoint of 1500 mm³ tumor size (FIG. 11B).

FIGS. 12A-12D include data showing immune cell infiltrates in s.c. MC38/CEA tumors in CEATg mice. Subcutaneous MC38/CEA tumors were collected from MC38/CEA tumor-bearing mice treated with i.p. injection of CY (100 mg/kg) followed by i.v. injection of CAR T cell therapy three days after T cell therapy (FIG. 12A) and at the termination timepoint of 1500 mm³ tumor size (FIG. 12B). The collected tumors were analyzed by immunohistochemistry (IHC) for T cells (CD3), B cells (CD19), macrophages (F4/80), natural killer cells (NKp46), and neutrophils (Ly6G). FIGS. 12C-12D include graphs showing quantification of CD3⁺ and/or CD19⁺ T cells.

FIGS. 13A-13D include data showing therapeutic efficacy of anti-CEA-IL2 immunocytokine (ICK) on CY plus anti-CEA CAR T cells on s.c. MC38/CEA tumor in CEATg mice. Subcutaneous MC38/CEA (1×10⁶) tumor-bearing CEATg mice were treated on day 11 post tumor implantation with i.p. injection of CY (100 mg/kg) followed by i.v. injection of either 5×10⁶ mock or anti-CEA CART cells on day 12 and i.p. injection of anti-CEA-IL-2 ICK (25 μg/mouse) on day 13. Due to tumor ulceration, mice had to be euthanized before reaching 1500 mm³. Individual mouse from each treatment group was graphed: (FIG. 13A) CY only treated group, (FIG. 13B) CY and ICK treated group, (FIG. 13C) CY and anti-CEA CAR T cells treated group, and (FIG. 13D) CY, anti-CEA CAR T cells, and ICK treated group. By day 36, 1/6 mice treated with CY and ICK; 2/8 mice treated with CY and CAR T cells; and 2/8 mice treated with CY, CAR T cells, and ICK had complete regression. Two additional mice treated with CY, CAR T cells, and ICK showed response.

FIGS. 14A-14E include data showing immune cell infiltrate phenotype in E0771/CEA tumor in CEATg mouse. Orthotopic E0771/CEA tumors and tumor-draining lymph node (TDLN) were collected from tumor-bearing CEATg mice treated with different combinations of CY, T cell therapy, and/or anti-CEA-IL-2 ICK when the tumor got to 1500 mm³. Lymphocytes in the tumor and TDLN were analyzed by flow cytometry for (FIG. 14A) FoxP3⁺ regulatory T cells, (FIG. 14B) CD4⁺ T cells, (FIG. 14C) CD8⁺ T cells, (FIG. 14D) IFN⁺ and/or PD1⁺ CD8⁺ T cells, and (FIG. 14E) IFN⁺ and/or PD1⁺ CD4+ T cells. (n=4 per group). ****p<0.0001; ***p<0.001; **p<0.01; *p<0.05.

The details of one or more embodiments of the invention are set forth in the description below. Other features or advantages of the present invention will be apparent from the drawings and detailed description of several embodiments, and also from the appended claims.

### DETAILED DESCRIPTION

Colon cancer is the third leading cause of cancer-related deaths in men and women, and breast cancer is the second most common cancer for women in the United States. See, e.g., MayoClinic, Colon Cancer; Colon Cancer Treatment (PDQ(R)): Health Professional Version, in PDQ Cancer Information Summaries. 2002: Bethesda Md.; and Bray et al. Global cancer statistics 2018: GLOBOCAN estimates of incidence and mortality worldwide for 36 cancers in 185 countries. Cancer J Clin. 2018; 68(6):394-424. The advent of targeted immunotherapy has played a major role in new approaches to the treatment of these cancers while reducing side effects associated with untargeted therapies. In terms of antibody-based tumor targeting, a potential target for both colon and breast cancers is carcinoembryonic antigen (CEA). CEA is expressed in over 90% of colon and about 50% of breast cancers; and high serum CEA levels have been correlated with poor prognosis in both colon and breast cancer patients. See, e.g., Gold & Freedman. Demonstration of tumor-specific antigens in human colonic carcinomata by immunological tolerance and absorption techniques. J Exp Med. 1965;121(3):439-462; and Molina et al. Tumor markers in breast cancer-european group on tumor markers recommendations. Tumour Biol. 2005; 26(6):281-293. Although CEA is expressed in normal colon, CEA expression is polarized to the apical side of epithelial cells and is not accessible to circulating antibodies; however, during tumor invasion, CEA expression on tumor cells becomes accessible to systemic antibodies as evidenced by targeting with radiolabeled anti-CEA antibodies.

Tumor antigens such as CEA that are expressed in normal colon are poorly immunogenic but can be retargeted by the use of chimeric antigen receptors (CARs) expressed on T cells since they are derived from monoclonal antibodies that were produced by immunization of mice with a human antigen. Similarly, endogenous T cells do not target CEA, but CAR T cells break tolerance by using CARs derived from monoclonal antibodies and bypass the requirement for antigen presentation in the context of MHC 31 Nonetheless, CAR T therapy alone for solid tumors is relatively ineffective and requires additional support.

Surprisingly, the anti-CEA CAR T cells disclosed herein in combination with the anti-CEA-IL2 immunocytokine (ICK) disclosed herein successfully reduced tumor burden in subcutaneous breast and colon cancer xenograft models and displayed long-term in vivo efficacy that prevented tumor growth after re-exposure to tumor cells. Significant reductions in tumor burden were also observed after treatment with cyclophosphamide (CY) followed by multiple administrations of the anti-CEA-IL ICK.

Accordingly, the present disclosure provides, in some aspects, therapeutic uses of anti-CEA CAR T cells and anti-CEA-IL-2 ICK for treating cancers in which CEA is highly expressed (e.g., colon cancer, breast cancer).
I. Anti-CEA CAR T Cells

A chimeric antigen receptor (CAR) refers to an artificial immune cell receptor that is engineered to recognize and bind to an antigen expressed by diseased cells such as cancer cells. A T cell that expresses a CAR polypeptide is referred to as a CAR T cell. CARs have the ability to redirect T-cell specificity and reactivity toward a selected target in a non-MHC-restricted manner. The non-MHC-restricted antigen recognition gives CAR T cells the ability to recognize an antigen independent of antigen processing, thereby bypassing a major mechanism of tumor escape.

There are various generations of CARs, each of which contains different components. First generation CARs join an antibody-derived scFv to the CD3ζ intracellular signaling domain of the T cell receptor through a spacer region (also called a hinge domain) and a transmembrane domain. Second generation CARs incorporate an additional co-stimulatory domain (e.g., CD28, 4-BB, or ICOS) to supply a co-stimulatory signal. Third generation CARs contain two co-stimulatory domains (e.g., a combination of CD27, CD28, 4-1BB, ICOS, or OX40) fused with the TCR CD3ζ chain. Any generation of CAR is within the scope of the present disclosure.

Generally, a CAR is a fusion polypeptide comprising an extracellular domain that recognizes a target antigen (e.g., a single chain fragment (scFv) of an antibody or other antibody fragment) and an intracellular domain comprising a signaling domain of the T cell receptor (TCR) complex (e.g., CD3ζ) and, in many cases, a co-stimulatory domain. A CAR can further comprise a spacer region and a transmembrane domain between the extracellular domain and the intracellular domain, and a signal peptide at the N-terminus for surface expression. Examples of signal peptides include MLLLVTSLLLCELPHPAFLLIP (SEQ ID NO: 1) and MALPVTALLLPLALLLHAARP (SEQ ID NO: 2). Other signal peptides can be used.

Provided herein are carcinoembryonic antigen (CEA) targeted CARs (also called “CEA CAR”) or CEA targeted polypeptide (also called “CEA polypeptide”) for treating cancer. The CEA CAR can comprise an anti-CEA scFv or an anti-CEA Fab, followed by a spacer region (e.g., IgG4, CD8, CD28, or a combination thereof) and a transmembrane domain (e.g., a CD28 transmembrane domain) that is fused to an intracellular signaling domain (e.g., CD3).

For example, when the CAR comprises an anti-CEA Fab followed by IgG4A, CD28, and CD3, the CAR targeted to CEA comprises the amino acid sequence:

(SEQ ID NO: 66)
METDTLLLWVLLLWVPGSTGDIQLTQSPSSLSASVGDRVTITCRAGESV
DIFGVGFLHWYQQKPGKAPKLLIYRASNLESGVPSRFSGSGSRTDFTLT
ISSLQPEDFATYYCQQTNEDPYTFGQGTKVEIKRTVAAPSVFIFPPSDE
QLKSGAASVVCLLNNFYPREAKVQWKVDNALQSGNSQESVTEQDSKDST
YSLSSTLTLSKADYEKHKVYACEVTHQGLSSPVTKSFNRGECGGGGSGG
GGSGGGGSGGGGSGGGGSGGGGSGGEVQLVESGGGLVQPGGSLRLSCAA
SGFNIKDTYMHWVRQAPGKGLEWVARIDPANGNSKYADSVKGRFTISAD
TSKNTAYLQMNSLRAEDTAVYYCAPFGYYVSDYAMAYWGQGTLVTVSSA
STKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVE
PKSCDKTHTESKYGPPCPPCPGGGSSGGGSGGQPREPQVYTLPPSQEEM
TKNQVSLTCLVKGFYPSDIAVEWESNGQPENNYKTTPPVLDSDGSFFLY
SRLTVDKSRWQEGNVFSCSVMHEALHNHYTQKSLSLSLGKMFWVLVVVG
GVLACYSLLVTVAFIIFWVRSKRSRGGHSDYMNMTPRRPGPTRKHYQPY
APPRDFAAYRSGGGRVKFSRSADAPAYQQGQNQLYNELNLGRREEYDVL
DKRRGRDPEMGGKPRRKNPQEGLYNELQKDKMAEAYSEIGMKGERRRGK
GHDGLYQGLSTATKDTYDALHMQALPPR with up to 5 or up
to 10 single amino acid substitutions.

In another example, when the CAR comprises an anti-CEA Fab followed by IgG4Δ, CD4, 4-1BB, and CD3ζ, the CAR targeted to CEA comprises the amino acid sequence:

(SEQ ID NO: 67)
METDTLLLWVLLLWVPGSTGDIQLTQSPSSLSASVGDRVTITCRAGESV
DIFGVGFLHWYQQKPGKAPKLLIYRASNLESGVPSRFSGSGSRTDFTLT
ISSLQPEDFATYYCQQTNEDPYTFGQGTKVEIKRTVAAPSVFIFPPSDE
QLKSGAASVVCLLNNFYPREAKVQWKVDNALQSGNSQESVTEQDSKDST
YSLSSTLTLSKADYEKHKVYACEVTHQGLSSPVTKSFNRGECGGGGSGG
GGSGGGGSGGGGSGGGGSGGGGSGGEVQLVESGGGLVQPGGSLRLSCAA
SGFNIKDTYMHWVRQAPGKGLEWVARIDPANGNSKYADSVKGRFTISAD
TSKNTAYLQMNSLRAEDTAVYYCAPFGYYVSDYAMAYWGQGTLVTVSSA
STKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVE
PKSCDKTHTESKYGPPCPPCPGGGSSGGGSGGQPREPQVYTLPPSQEEM
TKNQVSLTCLVKGFYPSDIAVEWESNGQPENNYKTTPPVLDSDGSFFLY
SRLTVDKSRWQEGNVFSCSVMHEALHNHYTQKSLSLSLGKMALIVLGGV
AGLLLFIGLGIFFKRGRKKLLYIFKQPFMRPVQTTQEEDGCSCRFPEEE
EGGCELGGGRVKFSRSADAPAYQQGQNQLYNELNLGRREEYDVLDKRRG
RDPEMGGKPRRKNPQEGLYNELQKDKMAEAYSEIGMKGERRRGKGHDGL
YQGLSTATKDTYDALHMQALPPRRVKFSRSADAPAYQQGQNQLYNELNL
GRREEYDVLDKRRGRDPEMGGKPRRKNPQEGLYNELQKDKMAEAYSEIG
MKGERRRGKGHDGLYQGLSTATKDTYDALHMQALPPR with up to
5 or up to 10 single amino acid substitutions.

In some examples, the CAR open reading frame is followed by a T2A ribosome skip sequence and a truncated CD19R (also called CD19t). In this arrangement, co-expression of CD19t provides an inert, non-immunogenic surface marker that allows for accurate measurement of gene modified cells, and enables positive selection of gene-modified cells, as well as efficient cell tracking of the therapeutic T cells in vivo following adoptive transfer. In some examples, the CAR does not include the T2A ribosome skip sequence and CD19t.

For example, when the CAR comprises an anti-CEA Fab followed by IgG4A, CD28, and CD3, and the CAR open reading frame is followed by a T2A ribosome skip sequence and a truncated CD19R, the amino acid sequence comprises:

(SEQ ID NO: 68)
METDTLLLWVLLLWVPGSTGDIQLTQSPSSLSASVGDRVTITCRAGESV
DIFGVGFLHWYQQKPGKAPKLLIYRASNLESGVPSRFSGSGSRTDFTLT
ISSLQPEDFATYYCQQTNEDPYTFGQGTKVEIKRTVAAPSVFIFPPSDE
QLKSGAASVVCLLNNFYPREAKVQWKVDNALQSGNSQESVTEQDSKDST
YSLSSTLTLSKADYEKHKVYACEVTHQGLSSPVTKSFNRGECGGGGSGG
GGSGGGGSGGGGSGGGGSGGGGSGGEVQLVESGGGLVQPGGSLRLSCAA
SGFNIKDTYMHWVRQAPGKGLEWVARIDPANGNSKYADSVKGRFTISAD
TSKNTAYLQMNSLRAEDTAVYYCAPFGYYVSDYAMAYWGQGTLVTVSSA
STKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVE
PKSCDKTHTESKYGPPCPPCPGGGSSGGGSGGQPREPQVYTLPPSQEEM
TKNQVSLTCLVKGFYPSDIAVEWESNGQPENNYKTTPPVLDSDGSFFLY
SRLTVDKSRWQEGNVFSCSVMHEALHNHYTQKSLSLSLGKMFWVLVVVG
GVLACYSLLVTVAFIIFWVRSKRSRGGHSDYMNMTPRRPGPTRKHYQPY
APPRDFAAYRSGGGRVKFSRSADAPAYQQGQNQLYNELNLGRREEYDVL
DKRRGRDPEMGGKPRRKNPQEGLYNELQKDKMAEAYSEIGMKGERRRGK
GHDGLYQGLSTATKDTYDALHMQALPPRLEGGGEGRGSLLTCGDVEENP
GPRMPPPRLLFFLLFLTPMEVRPEEPLVVKVEEGDNAVLQCLKGTSDGP
TQQLTWSRESPLKPFLKLSLGLPGLGIHMRPLAIWLFIFNVSQQMGGFY
LCQPGPPSEKAWQPGWTVNVEGSGELFRWNVSDLGGLGCGLKNRSSEGP
SSPSGKLMSPKLYVWAKDRPEIWEGEPPCVPPRDSLNQSLSQDLTMAPG
STLWLSCGVPPDSVSRGPLSWTHVHPKGPKSLLSLELKDDRPARDMWVM
ETGLLLPRATAQDAGKYYCHRGNLTMSFHLEITARPVLWHWLLRTGGWK
VSAVTLAYLIFCLCSLVGILHLQRALVLRRKR with up to 5
or up to 10 single amino acid substitutions.

In another example, when the CAR comprises an anti-CEA Fab followed by IgG4A, CD4, 4-1BB, and CD3ζ, and the CAR open reading frame is followed by a T2A ribosome skip sequence and a truncated CD19R, the amino acid sequence comprises:

(SEQ ID NO: 69)
METDTLLLWVLLLWVPGSTGDIQLTQSPSSLSASVGDRVTITCRAGESV
DIFGVGFLHWYQQKPGKAPKLLIYRASNLESGVPSRFSGSGSRTDFTLT
ISSLQPEDFATYYCQQTNEDPYTFGQGTKVEIKRTVAAPSVFIFPPSDE
QLKSGAASVVCLLNNFYPREAKVQWKVDNALQSGNSQESVTEQDSKDST
YSLSSTLTLSKADYEKHKVYACEVTHQGLSSPVTKSFNRGECGGGGSGG
GGSGGGGSGGGGSGGGGSGGGGSGGEVQLVESGGGLVQPGGSLRLSCAA
SGFNIKDTYMHWVRQAPGKGLEWVARIDPANGNSKYADSVKGRFTISAD
TSKNTAYLQMNSLRAEDTAVYYCAPFGYYVSDYAMAYWGQGTLVTVSSA
STKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVE
PKSCDKTHTESKYGPPCPPCPGGGSSGGGSGGQPREPQVYTLPPSQEEM
TKNQVSLTCLVKGFYPSDIAVEWESNGQPENNYKTTPPVLDSDGSFFLY
SRLTVDKSRWQEGNVFSCSVMHEALHNHYTQKSLSLSLGKMALIVLGGV
AGLLLFIGLGIFFKRGRKKLLYIFKQPFMRPVQTTQEEDGCSCRFPEEE
EGGCELGGGRVKFSRSADAPAYQQGQNQLYNELNLGRREEYDVLDKRRG
RDPEMGGKPRRKNPQEGLYNELQKDKMAEAYSEIGMKGERRRGKGHDGL
YQGLSTATKDTYDALHMQALPPRRVKFSRSADAPAYQQGQNQLYNELNL
GRREEYDVLDKRRGRDPEMGGKPRRKNPQEGLYNELQKDKMAEAYSEIG
MKGERRRGKGHDGLYQGLSTATKDTYDALHMQALPPRLEGGGEGRGSLL
TCGDVEENPGPRMPPPRLLFFLLFLTPMEVRPEEPLVVKVEEGDNAVLQ
CLKGTSDGPTQQLTWSRESPLKPFLKLSLGLPGLGIHMRPLAIWLFIFN
VSQQMGGFYLCQPGPPSEKAWQPGWTVNVEGSGELFRWNVSDLGGLGCG
LKNRSSEGPSSPSGKLMSPKLYVWAKDRPEIWEGEPPCVPPRDSLNQSL
SQDLTMAPGSTLWLSCGVPPDSVSRGPLSWTHVHPKGPKSLLSLELKDD
RPARDMWVMETGLLLPRATAQDAGKYYCHRGNLTMSFHLEITARPVLWH
WLLRTGGWKVSAVTLAYLIFCLCSLVGILHLQRALVLRRKR with up
to 5 or up to 10 single amino acid substitutions.

Human CEA (human sequence carcinoembryonic antigen-related cell adhesion molecule 5 isoform 1; GenBank NP 004354) has the sequence (amino acids 35-685 are the mature protein):

| (SEQ ID NO: 3) | |
|---|---|
| 1 | mespsapphr wcipwqrlll taslltfwnp pttaklties tpfnvaegke vlllvhnlpq |
| 61 | hlfgyswykg ervdgnrqii gyvigtqqat pgpaysgrei iypnaslliq niiqndtgfy |
| 121 | tlhviksdlv neeatgqfrv ypelpkpsis snnskpvedk davaftcepe tqdatylwwv |
| 181 | nnqslpvspr lqlsngnrtl tlfnvtrndt asykcetqnp vsarrsdsvi lnvlygpdap |
| 241 | tisplntsyr sgenlnlsch aasnppaqys wfvngtfqqs tqelfipnit vnnsgsytcq |
| 301 | ahnsdtglnr ttvttitvya eppkpfitsn nsnpvededa valtcepeiq nttylwwvnn |
| 361 | qslpvsprlq lsndnrtltl lsvtrndvgp yecgiqnels vdhsdpviln vlygpddpti |
| 421 | spsytyyrpg vnlslschaa snppaqyswl idgniqqhtq elfisnitek nsglytcqan |
| 481 | nsasghsrtt vktitvsael pkpsissnns kpvedkdava ftcepeaqnt tylwwvngqs |
| 541 | lpvsprlqls ngnrtltlfn vtrndarayv cgiqnsvsan rsdpvtldvl ygpdtpiisp |
| 601 | pdssylsgan lnlschsasn pspqyswrin gipqqhtqvl fiakitpnnn gtyacfvsnl |
| 661 | atgrnnsivk sitvsasgts pglsagatvg imigvlvgva li |

(a) Antigen Binding Extracellular Domain

The antigen binding extracellular domain is the region of a CAR polypeptide that is exposed to the extracellular fluid when the CAR is expressed on the cell surface. The antigen binding extracellular domain is specific to a target antigen of interest such as a tumor antigen (e.g., CEA).

In some examples, the antigen binding domain comprises a scFv, which includes an antibody heavy chain variable region (V_{H}) and an antibody light chain variable region (V_{L}). The scFV fragment retains the antigen binding specificity of the parent antibody, from which the scFv fragment is derived. The V_{H} and V_{L} domains can be in either orientation (i.e., V_{H}-V_{L} or V_{L}-V_{H}). In some examples, the V_{H} and V_{L} are linked via a peptide linker, which can include hydrophilic residues with stretches of glycine and serine for flexibility as well as stretches of glutamate and lysine for improved solubility. In some embodiments, the scFv can comprise humanized V_{H} and/or V_{L} domains. In some examples, a signal peptide can be located at the N-terminus to facilitate cell surface expression.

In the case of scFv targeted to human CEA it is desirable for the scFv to bind to the A3 domain of CEA (e.g., amino acids 501 to 588 of SEQ ID NO: 3, which is provided as SEQ ID NO: 4)

In some embodiments, the scFv targeted to CEA comprises the amino acid sequence:

(SEQ ID NO: 5)
DIVLTQSPASLAVSLGQRATMSCRAGESVDIFGVGFLHWYQQKPGQPPK
LLIYRASNLESGIPVRFSGTGSRTDFTLIIDPVEADDVATYYCQQTNED
PYTFGGGTKLEIKGSTSGGGSGGGSGGGGSSEVQLQQSGAELVEPGASV
KLSCTASGFNIKDTYMHWVKQRPEQGLEWIGRIDPANGNSKYVPKFQGK
ATITADTSSNTAYLQLTSLTSEDTAVYYCAPFGYYVSDYAMAYWGQGTS
VTVSSTTTK with up to 5 or up to 10 single amino
acid substitutions.

In some examples, the antigen binding domain comprises an antigen-binding fragment (Fab), which includes an antibody heavy chain variable region (V_{H}), an antibody heavy chain constant region (C_{H}), an antibody light chain variable region (V_{L}), and an antibody light chain constant region (C_{L}). The Fab retains the antigen binding specificity of the parent antibody, from which the Fab is derived. The V_{H}-C_{H}1 and V_{L}-C_{L} domains can be in either orientation (i.e., V_{H}-C_{H}1-V_{L}-C_{L} or V_{L}-C_{L}-V_{H}-C_{H}1). In some examples, the V_{H}-C_{H}1 and V_{L}-C_{L} are linked via a peptide linker, which can include hydrophilic residues with stretches of glycine and serine for flexibility as well as stretches of glutamate and lysine for improved solubility. In some embodiments, the Fab can comprise humanized V_{H} and/or V_{L} domains. In some examples, a signal peptide can be located at the N-terminus to facilitate cell surface expression.

In the case of Fab targeted to human CEA it is desirable for the Fab to bind to the A3 domain of CEA (e.g., amino acids 501 to 588 of SEQ ID NO: 3, which is provided as SEQ ID NO: 4).

In some embodiments, the Fab targeted to CEA comprises the amino acid sequence:

(SEQ ID NO: 65)
METDTLLLWVLLLWVPGSTGDIQLTQSPSSLSASVGDRVTITCRAGESV
DIFGVGFLHWYQQKPGKAPKLLIYRASNLESGVPSRFSGSGSRTDFTLT
ISSLQPEDFATYYCQQTNEDPYTFGQGTKVEIKRTVAAPSVFIFPPSDE
QLKSGAASVVCLLNNFYPREAKVQWKVDNALQSGNSQESVTEQDSKDST
YSLSSTLTLSKADYEKHKVYACEVTHQGLSSPVTKSFNRGECGGGGSGG
GGSGGGGSGGGGSGGGGSGGGGSGGEVQLVESGGGLVQPGGSLRLSCAA
SGFNIKDTYMHWVRQAPGKGLEWVARIDPANGNSKYADSVKGRFTISAD
TSKNTAYLQMNSLRAEDTAVYYCAPFGYYVSDYAMAYWGQGTLVTVSSA
STKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVE
PKSCDKTHT with up to 5 or up to 10 single amino
acid substitutions.

An amino acid modification refers to an amino acid substitution, insertion, and/or deletion in a protein or peptide sequence. An “amino acid substitution” or “substitution” refers to replacement of an amino acid at a particular position in a parent peptide or protein sequence with another amino acid. A substitution can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The following are examples of various groupings of amino acids: 1) Amino acids with nonpolar R groups: Alanine, Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

In certain embodiments, the CEA scFv comprises a light chain variable region that is at least 95% identical to or includes up to 5 single amino acid substitutions compared to: DIQLTQSPSSLSASVGDRVTITCRAGESVDIFGVGFLHWYQQKPGKAPKLLIYRASNL ESGVPSRFSGSGSRTDFTLTISSLQPEDFATYYCQQTNEDPYTFGQGTKVEIK (SEQ ID NO: 6). In certain embodiments, the CEA scFv comprises a light chain variable region that comprises a CDR1 comprising: RAGESVDIFGVGFLH (SEQ ID NO: 7), a CDR2 comprising RASNLES (SEQ ID NO: 8); and a CDR3 comprising QQTNEDPYT (SEQ ID NO: 9) and overall has no more than 10 (e.g., no more than 9, no more than 8, no more than 7, no more than 6, no more than 5, no more than 4, no more than 3, no more than 2, or no more than 1) single amino acid substitutions compared to SEQ ID NO: 6. In certain embodiments, the CEA scFv comprises a heavy chain variable region that is at least 95% identical to or includes up to 5 single amino acid substitutions compared to: EVQLVESGGGLVQPGGSLRLSCAASGFNIKDTYMHWVRQAPGKGLEWVARIDPAN GNSKYADSVKGRFTISADTSKNTAYLQMNSLRAEDTAVYYCAPFGYYVSDYAMAY WGQG (SEQ ID NO: 10). In certain embodiments, the CEA scFv comprises a heavy chain variable region that comprises a CDR1 comprising: DTYMH (SEQ ID NO: 11), a CDR2 comprising RIDPANGNSKYADSVKG (SEQ ID NO: 12); and a CDR3 comprising FGYYVSDYAMAY (SEQ ID NO: 13) and overall has no more than 10 (e.g., no more than 9, no more than 8, no more than 7, no more than 6, no more than 5, no more than 4, no more than 3, no more than 2, or no more than 1) single amino acid substitutions compared to SEQ ID NO: 10.

In certain embodiments, the CEA scFv comprises a heavy chain variable region that is at least 95% identical to or includes up to 5 single amino acid substitutions compared to: EVQLVESGGGLVQPGGSLRLSCAASGFNIKDTYMHWVRQAPGKGLEWVARIDPAN GNSKYVPKFQGRATISADTSKNTAYLQMNSLRAEDTAVYYCAPFGYYVSDYAMAY WGQG (SEQ ID NO: 14). In certain embodiments, the CEA scFv comprises a heavy chain variable region that comprises a CDR1 comprising: DTYMH (SEQ ID NO: 11), a CDR2 comprising RIDPANGNSKYVPKFQG (SEQ ID NO: 15); and a CDR3 comprising FGYYVSDYAMAY (SEQ ID NO: 13) and overall has no more than 10 (e.g., no more than 9, no more than 8, no more than 7, no more than 6, no more than 5, no more than 4, no more than 3, no more than 2, or no more than 1) single amino acid substitutions compared to SEQ ID NO: 14.

In certain embodiments, the CEA scFv comprises a light chain variable region comprising DIQLTQSPSSLSASVGDRVTITCRAGESVDIFGVGFLHWYQQKPGKAPKLLIYRASNL ESGVPSRFSGSGSRTDFTLTISSLQPEDFATYYCQQTNEDPYTFGQGTKVEIK (SEQ ID NO: 6) and a heavy chain variable region comprising EVQLVESGGGLVQPGGSLRLSCAASGFNIKDTYMHWVRQAPGKGLEWVARIDPAN GNSKYADSVKGRFTISADTSKNTAYLQMNSLRAEDTAVYYCAPFGYYVSDYAMAY WGQG (SEQ ID NO: 10) joined by a linker of 5-20 amino acids. In certain embodiments, the CEA scFv comprises a light chain variable region comprising DIQLTQSPSSLSASVGDRVTITCRAGESVDIFGVGFLHWYQQKPGKAPKLLIYRASNL ESGVPSRFSGSGSRTDFTLTISSLQPEDFATYYCQQTNEDPYTFGQGTKVEIK (SEQ ID NO: 6) and a heavy chain variable region comprising EVQLVESGGGLVQPGGSLRLSCAASGFNIKDTYMHWVRQAPGKGLEWVARIDPAN GNSKYADSVKGRFTISADTSKNTAYLQMNSLRAEDTAVYYCAPFGYYVSDYAMAY WGQG (SEQ ID NO: 14) joined by a linker of 5-20 amino acids. In some embodiments, a useful flexible linker is 1, 2, 3, 4, 5, 6, 7, 8, 9, or 10 repeats of the sequence GGGS (SEQ ID NO: 16). In some embodiments, a useful flexible linker is 1, 2, 3, 4, 5, 6, 7, 8, 9, or 10 repeats of the sequence GGGGS (SEQ ID NO: 17). In some embodiments, the light chain variable region is amino terminal to the heavy chain variable region.

In certain embodiments, the CEA Fab comprises a light chain variable region that is at least 95% identical to or includes up to 5 single amino acid substitutions compared to: METDTLLLWVLLLWVPGSTGDIQLTQSPSSLSASVGDRVTITCRAGESVDIFGVGFLH WYQQKPGKAPKLLIYRASNLESGVPSRFSGSGSRTDFTLTIS SLQPEDFATYYCQQTN EDPYTFGQGTKVEIK (SEQ ID NO: 61). In certain embodiments, the CEA Fab comprises a light chain variable region that comprises a CDR1 comprising: RAGESVDIFGVGFLH (SEQ ID NO: 7), a CDR2 comprising RASNLES (SEQ ID NO: 8); and a CDR3 comprising QQTNEDPYT (SEQ ID NO: 9) and overall has no more than 10 (e.g., no more than 9, no more than 8, no more than 7, no more than 6, no more than 5, no more than 4, no more than 3, no more than 2, or no more than 1) single amino acid substitutions compared to SEQ ID NO: 61.

In certain embodiments, the CEA Fab comprises a heavy chain variable region that is at least 95% identical to or includes up to 5 single amino acid substitutions compared to: EVQLVESGGGLVQPGGSLRLSCAASGFNIKDTYMHWVRQAPGKGLEWVARIDPAN GNSKYADSVKGRFTISADTSKNTAYLQMNSLRAEDTAVYYCAPFGYYVSDYAMAY WGQGTLVTVSS (SEQ ID NO: 63). In certain embodiments, the CEA scFv comprises a heavy chain variable region that comprises a CDR1 comprising: DTYMH (SEQ ID NO: 11), a CDR2 comprising RIDPANGNSKYADSVKG (SEQ ID NO: 12); and a CDR3 comprising FGYYVSDYAMAY (SEQ ID NO: 13) and overall has no more than 10 (e.g., no more than 9, no more than 8, no more than 7, no more than 6, no more than 5, no more than 4, no more than 3, no more than 2, or no more than 1) single amino acid substitutions compared to SEQ ID NO: 63.

In certain embodiments, the CEA Fab comprises a light chain constant region that is at least 95% identical to or includes up to 5 single amino acid substitutions compared to:

(SEQ ID NO: 62)
RTVAAPSVFIFPPSDEQLKSGAASVVCLLNNFYPREAKVQWKVDNALQS
GNSQESVTEQDSKDSTYSLSSTLTLSKADYEKHKVYACEVTHQGLSSPV
TKSFNRGEC.

In certain embodiments, the CEA Fab comprises a heavy chain constant region that is at least 95% identical to or includes up to 5 single amino acid substitutions compared to:

(SEQ ID NO: 64)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHT.

In certain embodiments, the CEA Fab comprises a light chain constant region comprising RTVAAPSVFIFPPSDEQLKSGAASVVCLLNNFYPREAKVQWKVDNALQSGNSQESVT EQDSKDSTYSLSSTLTLSKADYEKHKVYACEVTHQGLSSPVTKSFNRGEC (SEQ ID NO: 62) and a heavy chain variable region comprising EVQLVESGGGLVQPGGSLRLSCAASGFNIKDTYMHWVRQAPGKGLEWVARIDPAN GNSKYADSVKGRFTISADTSKNTAYLQMNSLRAEDTAVYYCAPFGYYVSDYAMAY WGQGTLVTVSS (SEQ ID NO: 63) joined by a linker of 5-40 amino acids (e.g., 30 amino acids). In some embodiments, a useful flexible linker is 1, 2, 3, 4, 5, 6, 7, 8, 9, or 10 repeats of the sequence GGGS (SEQ ID NO: 16). In some embodiments, a useful flexible linker is 1, 2, 3, 4, 5, 6, 7, 8, 9, or 10 repeats of the sequence GGGGS (SEQ ID NO: 17). In some embodiments, the heavy chain constant region is amino terminal to the light chain variable region.

(b) Transmembrane Domain

The CAR polypeptides disclosed herein can contain a transmembrane domain, which can be a hydrophobic alpha helix that spans the membrane. As used herein, a transmembrane domain refers to any protein structure that is thermodynamically stable in a cell membrane, preferably a eukaryotic cell membrane.

The transmembrane domain of a CAR as provided herein can be a CD8 transmembrane domain, a CD28 transmembrane domain, or a chimera of a CD8 and a CD28 transmembrane domain. In some examples, the transmembrane domain is a CD28 transmembrane domain comprising the sequence of SKRSRLLHSDYMNMTPRRPGPTRKHYQPYAPPRDFAAYRS (SEQ ID NO: 18). Other transmembrane domains can be used including those shown below.

TABLE 1
Examples of Transmembrane Domains

| Name | Accession | Length | Sequence |
|---|---|---|---|
| CD3z | J04132.1 | 21 aa | LCYLLDGILFIYGVILTALFL |
| | | | (SEQ ID NO: 19) |
| CD28 | NM_006139 | 27aa | FWVLVVVGGVLACYSLLVTVAFIIFWV |
| | | | (SEQ ID NO: 20) |
| CD28(M) | NM_006139 | 28aa | MFWVLVVVGGVLACYSLLVTVAFIIFWV |
| | | | (SEQ ID NO: 21) |
| CD4 | M35160 | 22aa | MALIVLGGVAGLLLFIGLGIFF |
| | | | (SEQ ID NO: 22) |
| CD8tm | NM_001768 | 21aa | IYIWAPLAGTCGVLLLSLVIT |
| | | | (SEQ ID NO: 23) |
| CD81m2 | NM_001768 | 23aa | IYIWAPLAGTCGVLLLSLVITLY |
| | | | (SEQ ID NO: 24) |
| CD81m3 | NM_001768 | 24aa | IYIWAPLAGTCGVLLLSLVITLYC |
| | | | (SEQ ID NO: 25) |
| 4-1BB | NM_001561 | 27aa | IISFFLALTSTALLFLLFFLTLRFSW |
| | | | (SEQ ID NO: 26) |
| NKG2D | NM_007360 | 21aa | PFFFCCFIAVAMGIRFIIMVA |
| | | | (SEQ ID NO: 27) |

(c) Spacer Region

The CAR or polypeptide described herein can include a spacer region located between the CEA targeting domain (i.e., a CEA targeted scFv or variant thereof) and the transmembrane domain. The spacer region can function to provide flexibility to the CAR, or domains thereof, or to prevent steric hindrance of the CAR, or domains thereof. A variety of different spacers can be used. Some of them include at least portion of a human Fc region, for example a hinge portion of a human Fc region or a CH3 domain or variants thereof. Table 2 below provides various spacers that can be used in the CARs described herein.

TABLE 2
Examples of Spacers

| Name | Length | Sequence |
|---|---|---|
| a3 | 3 aa | AAA |
| linker | 10 aa | GGGSSGGGSG (SEQ ID NO: 28) |
| IgG4 hinge (S→P) | 12 aa | ESKYGPPCPPCP (SEQ ID NO: 29) |
| (S228P) | | |
| IgG4 hinge | 12 aa | ESKYGPPCPSCP (SEQ ID NO: 30) |
| IgG4 hinge (S228P) + linker | 22 aa | ESKYGPPCPPCPGGGSSGGGSG (SEQ ID NO: 31) |
| CD28 hinge | 39 aa | IEVMYPPPYLDNEKSNGTIIHVKGKHLCPSPLFPGPSKP |
| | | (SEQ ID NO: 32) |
| CD8 hinge-48aa | 48 aa | AKPTTTPAPRPPTPAPTIASQPLSLRPEACRPAAGGAVHTR |
| | | GLDFACD (SEQ ID NO: 33) |
| CD8 hinge-45aa | 45aa | TTTPAPRPPTPAPTIASQPLSLRPEACRPAAGGAVHTRGLD |
| | | FACD (SEQ ID NO: 34) |
| IgG4(HL-CH3) | 129 aa | ESKYGPPCPPCPGGGSSGGGSGGQPREPQVYTLPPSQEEMT |
| Also called IgG4(HL-ΔCH2) | | KNQVSLTCLVKGFYPSDIAVEWESNGQPENNYKTTPPVLDS |
| (includes S228P in hinge) | | DGSFFLYSRLTVDKSRWQEGNVFSCSVMHEALHNHYTQKSL |
| | | SLSLGK (SEQ ID NO: 35) |
| IgG4(L235E, N297Q) | 229 aa | ESKYGPPCPSCPAPEFEGGPSVFLFPPKPKDTLMISRTPEV |
| | | TCVVVDVSQEDPEVQFNWYVDGVEVHNAKTKPREEQFQSTY |
| | | RVVSVLTVLHQDWLNGKEYKCKVSNKGLPSSIEKTISKAKG |
| | | QPREPQVYTLPPSQEEMTKNQVSLTCLVKGFYPSDIAVEWE |
| | | SNGQPENNYKTTPPVLDSDGSFFLYSRLTVDKSRWQEGNVF |
| | | SCSVMHEALHNHYTQKSLSLSLGK |
| | | (SEQ ID NO: 36) |
| IgG4(S228P, L235E, N297Q) | 229 aa | ESKYGPPCPPCPAPEFEGGPSVFLFPPKPKDTLMISRTPEV |
| | | TCVVVDVSQEDPEVQFNWYVDGVEVHNAKTKPREEQFQSTY |
| | | RVVSVLTVLHQDWLNGKEYKCKVSNKGLPSSIEKTISKAKG |
| | | QPREPQVYTLPPSQEEMTKNQVSLTCLVKGFYPSDIAVEWE |
| | | SNGQPENNYKTTPPVLDSDGSFFLYSRLTVDKSRWQEGNVF |
| | | SCSVMHEALHNHYTQKSLSLSLGK |
| | | (SEQ ID NO: 37) |
| IgG4(CH3) | 107 aa | GQPREPQVYTLPPSQEEMTKNQVSLTCLVKGFYPSDIAVEW |
| Also called IgG4(ΔCH2) | | ESNGQPENNYKTTPPVLDSDGSFFLYSRLTVDKSRWQEGNV |
| | | FSCSVMHEALHNHYTQKSLSLSLGK |
| | | (SEQ ID NO: 38) |

Some spacer regions include all or part of an immunoglobulin (e.g., IgG1, IgG2, IgG3, IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain (called CH3 or ACH2) or both a CH3 domain and a CH2 domain. The immunoglobulin derived sequences can include one or more amino acid modifications, for example, 1, 2, 3, 4 or 5 substitutions, e.g., substitutions that reduce off-target binding.

The hinge/linker region can also comprise an IgG4 hinge region having the sequence ESKYGPPCPSCP (SEQ ID NO: 30) or ESKYGPPCPPCP (SEQ ID NO: 29). The hinge/linger region can also comprise the sequence ESKYGPPCPPCP (SEQ ID NO: 29) followed by the linker sequence GGGSSGGGSG (SEQ ID NO: 28) followed by IgG4 CH3 sequence GQPREPQVYTLPPSQEEMTKNQVSLTCLVKGFYPSDIAVEWESNGQPENNYKTTPPV LDSDGSFFLYSRLTVDKSRWQEGNVFSCSVMHEALHNHYTQKSLSLSLGK (SEQ ID NO: 38). Thus, the entire linker/spacer region can comprise the sequence: ESKYGPPCPPCPGGGSSGGGSGGQPREPQVYTLPPSQEEMTKNQVSLTCLVKGFYPS DIAVEWESNGQPENNYKTTPPVLDSDGSFFLYSRLTVDKSRWQEGNVFSCSVMHEA LHNHYTQKSLSLSLGK (SEQ ID NO: 35). In some cases, the spacer has 1, 2, 3, 4, or 5 single amino acid changes (e.g., conservative changes) compared to SEQ ID NO: 31. In some cases, the IgG4 Fc hinge/linker region that is mutated at two positions (L235E; N297Q) in a manner that reduces binding by Fc receptors (FcRs).

(d) Intracellular Signaling Domains

Any of the CAR constructs described herein contain one or more intracellular signaling domains (e.g., CD3, and optionally one or more co-stimulatory domains), which are the functional end of the receptor. Following antigen recognition, receptors cluster and a signal is transmitted to the cell.

CD3ζ is the cytoplasmic signaling domain of the T cell receptor complex. CD3ζ contains three immunoreceptor tyrosine-based activation motifs (ITAMs), which transmit an activation signal to the T cell after the T cell is engaged with a cognate antigen. In some cases, CD3ζ provides a primary T cell activation signal but not a fully competent activation signal, which requires a co-stimulatory signal.

Accordingly, in some examples, the CAR polypeptides disclosed herein may further comprise one or more co-stimulatory signaling domains in addition to CD3ζ. For example, the co-stimulatory domain CD28 and/or 4-1BB can be used to transmit a proliferative/survival signal together with the primary signaling mediated by CD3ζ.

The co-stimulatory domain(s) are located between the transmembrane domain and the CD3ζ signaling domain. Table 3 includes examples of suitable co-stimulatory domains together with the sequence of the CD3ζ signaling domain.

TABLE 3
CD35 Domain and Examples of Co-stimulatory Domains

| Name | Accession | Length | Sequence |
|---|---|---|---|
| | J04132.1 | 113 aa | RVKFSRSADAPAYQQGQNQLYNELNLGRREEYDVLD |
| CD3ζ | | | KRRGRDPEMGGKPRRKNPQEGLYNELQKDKMAEAYS |
| | | | EIGMKGERRRGKGHDGLYQGLSTATKDTYDALHMQA |
| | | | LPPR (SEQ ID NO: 39) |
| CD3ζ | | 113 aa | IT AMS 1-3 underlined |
| variant | | | RVKFSRSADAPAYQQGQNQLYNELNLGRREEYDVLD |
| | | | KRRGRDPEMGGKPRRKNPQEGLFNELQKDKMAEAFS |
| | | | EIGMKGERRRGKGHDGLFQGLSTATKDTFDALHMQA |
| | | | LPPR (SEQ ID NO: 40) |
| CD3ζ | | 113 aa | RVKFSRSADAPAYQQGQNQLYNELNLGRREEYDVLD |
| variant | | | KRRGRDPEMGGKPRRKNPQEGLFNELQKDKMAEAFS |
| | | | EIGMKGERRRGKGHDGLYQGLSTATKDTYDALHMQA |
| | | | LPPR (SEQ ID NO: 41) |
| CD3ζ | | 113 aa | RVKFSRSADAPAYQQGQNQLYNELNLGRREEYDVLD |
| variant | | | KRRGRDPEMGGKPRRKNPQEGLYNELQKDKMAEAYS |
| | | | EIGMKGERRRGKGHDGLFQGLSTATKDTFDALHMQA |
| | | | LPPR (SEQ ID NO: 42) |
| CD3ζ | | 113 aa | RVKFSRSADAPAYQQGQNQLYNELNLGRREEYDVLD |
| variant | | | KRRGRDPEMGGKPRRKNPQEGLYNELQKDKMAEAFS |
| | | | EIGMKGERRRGKGHDGLFQGLSTATKDTFDALHMQA |
| | | | LPPR (SEQ ID NO: 43) |
| CD3ζ | | 113 aa | RVKFSRSADAPAYQQGQNQLYNELNLGRREEYDVLD |
| variant | | | KRRGRDPEMGGKPRRKNPQEGLFNELQKDKMAEAYS |
| | | | EIGMKGERRRGKGHDGLFQGLSTATKDTFDALHMQA |
| | | | LPPR (SEQ ID NO: 44) |
| CD3ζ | | | |
| variant | | 113 aa | RVKFSRSADAPAYQQGQNQLYNELNLGRREEYDVLD |
| | | | KRRGRDPEMGGKPRRKNPQEGLFNELQKDKMAEAFS |
| | | | EIGMKGERRRGKGHDGLYQGLSTATKDTFDALHMQA |
| | | | LPPR (SEQ ID NO: 45) |
| CD3ζ | | 113 aa | RVKFSRSADAPAYQQGQNQLYNELNLGRREEYDVLD |
| variant | | | KRRGRDPEMGGKPRRKNPQEGLFNELQKDKMAEAFS |
| | | | EIGMKGERRRGKGHDGLFQGLSTATKDTYDALHMQA |
| | | | LPPR (SEQ ID NO: 46) |
| CD28 | NM_006139 | 42 aa | RSKRSRLLH SD YMNMTPRRPGPTRKHYQPYAPPR |
| | | | DFAAYRS (SEQ ID NO: 47) |
| CD28gg* | NM_006139 | 42 aa | RSKRSRGGH SD YMNMTPRRPGPTRKHYQPYAPPR |
| | | | DFAAYRS (SEQ ID NO: 48) |
| 41BB | NM_001561 | 42 aa | KRGRKKLLYIFKQPFMRPVQTTQEEDGCSCRFPEEE |
| | | | EGGCEL (SEQ ID NO: 49) |
| OX40 | NM_003327 | 42 aa | ALYLLRRDQRLPPDAHKPPGGGSFRTPIQEEQADAH |
| | | | STLAKI (SEQ ID NO: 50) |
| 2B4 | NM_016382 | 120 aa | WRRKRKEKQSETSPKEFLTIYEDVKDLKTRRNHEQE |
| | | | QTFPGGGSTIYSMIQSQSSAPTSQEPAYTLYSLIQP |
| | | | SRKSGSRKRNHSPSFNSTIYEVIGKSQPKAQNPARL |
| | | | SRKELENFDVYS (SEQ ID NO: 51) |
| CTLA4 | NP_005205.2 | 223 aa | MACLGFQRHKAQLNLATRTWPCTLLFFLLFIPVFCK |
| | | | AMHVAQPAVVLASSRGIASFVCEYASPGKATEVRVT |
| | | | VLRQADSQVTEVCAATYMMGNELTFLDDSICTGTSS |
| | | | GNQVNLTIQGLRAMDTGLYICKVELMYPPPYYLGIG |
| | | | NGTQIYVIDPEPCPDSDFLLWILAAVSSGLFFYSFL |
| | | | LTAVSLSKMLKKRSPLTTGVYVKMPPTEPECEKQFQ |
| | | | PYFIPIN (SEQ ID NO: 52) |

In some examples, the CD3ζ signaling domain comprises an amino acid sequence that is at least 90%, at least 95%, at least 98% identical to SEQ ID NO: 39. In such instances, the CD3 signaling domain has 1, 2, 3, 4, or 5 amino acid changes (preferably conservative substitutions) compared to SEQ ID NO: 39. In other examples, the CD3ζ signaling domain is SEQ ID NO: 39.

In various embodiments: the co-stimulatory domain is selected from the group consisting of: a co-stimulatory domain depicted in Table 3 or a variant thereof having 1-5 (e.g., 1 or 2) amino acid modifications, a CD28 co-stimulatory domain or a variant thereof having 1-5 (e.g., 1 or 2) amino acid modifications, a 4-1BB co-stimulatory domain or a variant thereof having 1-5 (e.g., 1 or 2) amino acid modifications and an OX40 co-stimulatory domain or a variant thereof having 1-5 (e.g., 1 or 2) amino acid modifications. In certain embodiments, a 4-1BB co-stimulatory domain or a variant thereof having 1-5 (e.g., 1 or 2) amino acid modifications is present in the CAR polypeptides described herein.

In some embodiments, there are two co-stimulatory domains, for example, a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions). In various embodiments the 1-5 (e.g., 1 or 2) amino acid modification are substitutions. In various embodiments, the co-stimulatory domain is amino terminal to the CD3ζ signaling domain and a short linker consisting of 2 — 10, e.g., 3 amino acids (e.g., GGG) is can be positioned between the co-stimulatory domain and the CD3ζ signaling domain.
II. Immunocytokine (ICK) Therapy

While some anti-CEA antibodies have limited effects on CEA-positive tumors, such antibodies can be used to direct potent antitumor effects as an antibody-IL-2 fusion protein (immunocytokine, ICK), and at the same time reduce the toxicity of IL-2 as a single agent.

Accordingly, provided herein are anti-CEA-IL-2 ICK proteins comprising an anti-CEA antibody or antigen binding fragment thereof and IL-2. As used herein, “anti-CEA-IL-2 ICK” refers to an anti-CEA antibody or antigen binding fragment thereof in complex with IL-2, e.g., an anti-CEA antibody or antigen binding fragment thereof fused to IL-2. In such instances, IL-2 can be fused directly or indirectly via a linker or chemical conjugation to the heavy chain or a portion thereof (e.g., the heavy chain variable domain) or the light chain or a portion thereof (e.g., the light chain variable domain). In some examples, the heavy chain of the anti-CEA antibody fused at its carboxy terminus to IL-2, and the light chain variable region of the anti-CEA antibody is associated with the heavy chain, thereby forming a complex.

The anti-CEA-IL-2 ICK (also called ICK) provided herein can comprise a heavy chain variable domain sequence having 1-5 (e.g., 1 or 2) amino acid modifications (e.g., substitutions). In some examples, the ICK comprises a heavy chain variable domain sequence comprising: EVQLVESGGGLVQPGGSLRLSCAASGFNIKDTYMHWVRQAPGKGLEWVARIDPAN GNSKYADSVKGRFTISADTSKNTAYLQMNSLRAEDTAVYYCAPFGYYVSDYAMAY WGQG (SEQ ID NO: 10) and overall has no more than 10 (e.g., no more than 9, no more than 8, no more than 7, no more than 6, no more than 5, no more than 4, no more than 3, no more than 2, or no more than 1) single amino acid substitutions compared to SEQ ID NO: 10.

In some examples, the ICK comprises a heavy chain variable domain sequence comprising: EVQLVESGGGLVQPGGSLRLSCAASGFNIKDTYMHWVRQAPGKGLEWVARIDPAN GNSKYADSVKGRFTISADTSKNTAYLQMNSLRAEDTAVYYCAPFGYYVSDYAMAY WGQG (SEQ ID NO: 14) and overall has no more than 10 (e.g., no more than 9, no more than 8, no more than 7, no more than 6, no more than 5, no more than 4, no more than 3, no more than 2, or no more than 1) single amino acid substitutions compared to SEQ ID NO: 14.

The ICK provided herein can comprise a heavy chain sequence having 1-5 (e.g., 1 or 2) amino acid modifications (e.g., substitutions). In some examples, the ICK comprises a heavy chain sequence that is at least 90%, at least 95%, at least 98% identical to or identical to: EVQLVESGGGLVQPGGSLRLSCAASGFNIKDTYMHWVRQAPGKGLEWVARIDPAN GNSKYADSVKGRFTISADTSKNTAYLQMNSLRAEDTAVYYCAPFGYYVSDYAMAY WGQGTLVTVSSASTKGPSVFPLAPSSKSTSGGTAALGCLVKDYFPEPVTVSWNSGAL TSGVHTFPAVLQS SGLYSLSSVVTVPSSSLGTQTYICNVNHKPSNTKVDKKVEPKSCD KTHTCPPCPAPELLGGPSVFLFPPKPKDTLMISRTPEVTCVVVDVSHEDPEVKFNWYV DGVEVHNAKTKPREEQYNSTYRVVSVLTVLHQDWLNGKEYKCKVSNKALPAPIEK TISKAKGQPREPQVYTLPPSRDELTKNQVSLTCLVKGFYPSDIAVEWESNGQPENNY KTTPPVLDSDGSFFLYSKLTVDKSRWQQGNVFSCSVMHEALHNHYTQKSATATPG (SEQ ID NO: 53) and overall has no more than 10 (e.g., no more than 9, no more than 8, no more than 7, no more than 6, no more than 5, no more than 4, no more than 3, no more than 2, or no more than 1) single amino acid substitutions compared to SEQ ID NO: 53.

In certain embodiments, the ICK comprises a heavy chain variable region that is at least 95% identical to or includes up to 5 single amino acid substitutions compared to:

(SEQ ID NO: 10)
EVQLVESGGGLVQPGGSLRLSCAASGFNIKDTYMHWVRQAPGKGLEWVAR
IDPANGNSKYADSVKGRFTISADTSKNTAYLQMNSLRAEDTAVYYCAPFG
YYVSDYAMAYWGQG.

In certain embodiments, the ICK comprises a heavy chain variable region that comprises a CDR1 comprising: DTYMH (SEQ ID NO: 11), a CDR2 comprising RIDPANGNSKYADSVKG (SEQ ID NO: 12); and a CDR3 comprising FGYYVSDYAMAY (SEQ ID NO: 13) and overall has no more than 10 (e.g., no more than 9, no more than 8, no more than 7, no more than 6, no more than 5, no more than 4, no more than 3, no more than 2, or no more than 1) single amino acid substitutions compared to SEQ ID NO: 10).

In certain embodiments, the ICK comprises a heavy chain variable region that is at least 95% identical to or includes up to 5 single amino acid substitutions compared to: EVQLVESGGGLVQPGGSLRLSCAASGFNIKDTYMHWVRQAPGKGLEWVARIDPAN GNSKYVPKFQGRATISADTSKNTAYLQMNSLRAEDTAVYYCAPFGYYVSDYAMAY WGQG (SEQ ID NO: 14). In certain embodiments, the ICK comprises a heavy chain variable region that comprises a CDR1 comprising: DTYMH (SEQ ID NO: 11), a CDR2 comprising to RIDPANGNSKYVPKFQG (SEQ ID NO: 15); and a CDR3 comprising FGYYVSDYAMAY (SEQ ID NO: 13) and overall has no more than 10 (e.g., no more than 9, no more than 8, no more than 7, no more than 6, no more than 5, no more than 4, no more than 3, no more than 2, or no more than 1) single amino acid substitutions compared to SEQ ID NO: 14.

The ICK provided herein can comprise a light chain variable domain sequence having 1-5 (e.g., 1 or 2) amino acid modifications (e.g., substitutions). In some examples, the ICK comprises a light chain variable domain sequence that is at least 95% identical to or identical to: DIQLTQSPSSLSASVGDRVTITCRAGESVDIFGVGFLHWYQQKPGKAPKLLIYRASNL ESGVPSRFSGSGSRTDFTLTISSLQPEDFATYYCQQTNEDPYTFGQGTKVEIK (SEQ ID NO: 6) and overall has no more than 10 (e.g., no more than 9, no more than 8, no more than 7, no more than 6, no more than 5, no more than 4, no more than 3, no more than 2, or no more than 1) single amino acid substitutions compared to SEQ ID NO: 6.

The ICK provided herein can comprise a light chain sequence having 1-5 (e.g., 1 or 2) amino acid modifications (e.g., substitutions). In some examples, the ICK comprises a light chain sequence that is at least 95%, at least 98% identical to or identical to:

(SEQ ID NO: 54)
DIQLTQSPSSLSASVGDRVTITCRAGESVDIFGVGFLHWYQQKPGKAPKL
LIYRASNLESGVPSRFSGSGSRTDFTLTISSLQPEDFATYYCQQTNEDPY
TFGQGTKVEIKRTVAAPSVFIFPPSDEQLKSGAASVVCLLNNFYPREAKV
QWKVDNALQSGNSQESVTEQDSKDSTYSLSSTLTLSKADYEKHKVYACEV
THQGLSSPVTKSFNRGEC

and overall has no more than 10 (e.g., no more than 9, no more than 8, no more than 7, no more than 6, no more than 5, no more than 4, no more than 3, no more than 2, or no more than 1) single amino acid substitutions compared to SEQ ID NO: 54.

The ICK provided herein can comprise a full-length IL-2 or a fragment thereof. In some examples, the ICK comprises IL-2 or a variant thereof having 1-5 (e.g., 1 or 2) amino acid modifications (e.g., substitutions). In some examples, the ICK comprises IL-2 having a sequence that is at least 90%, at least 95%, at least 98% identical to or identical to:

(SEQ ID NO: 55)
MYRMQLLSCIALSLALVANSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTFMCEYADETATIVEFLNRWITFCQSIIS
TLT.

In some examples, the ICK comprises IL-2 having a sequence that is at least 90%, at least 95%, at least 98% identical to or identical to:

(SEQ ID NO: 56)
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT.

The anti-CEA antibody or antigen binding fragment thereof can be linked to IL-2 using any method known in the art. For example, the anti-CEA antibody or antigen binding fragment thereof can be linked to IL-2 via a polypeptide bond, e.g., a fusion protein comprising the heavy chain of the anti-CEA antibody fused at its carboxy terminus to IL-2. In another example, the anti-CEA antibody or antigen binding fragment thereof can be linked to IL-2 via reactive groups (e.g., sulfhydryl groups) within amino acid side chains of the anti-CEA antibody or antigen binding fragment thereof and IL-2. The anti-CEA antibody or antigen binding fragment thereof and IL-2 can be linked directly or indirectly, e.g., through a linker or a spacer.

The anti-CEA antibody or antigen binding fragment thereof can be linked to IL-2 in any order that does not interfere with binding of the antibody to the target. For example, the anti-CEA antibody or antigen binding fragment thereof can be linked to either the N- or C-terminus of IL-2. In another example, the anti-CEA antibody or antigen binding fragment thereof can be linked to an internal region of IL-2, or conversely, IL-2 can be linked to an internal region of the anti-CEA antibody or antigen binding fragment thereof.

The heavy chain of the anti-CEA antibody can be associated, either covalently or non-covalently, with the light chain of the anti-CEA antibody in the ICK proteins described herein.

The ICK described herein can comprise other modifications, e.g., modifications to increase serum half-life and/or bioavailability. For example, the ICK can comprise D amino acids, non-naturally occurring amino acids, and/or polyethylene glycol (PEG).
III. Preparation of Anti-CEA CAR and/or ICK T Cells

In some cases, the CEA CAR or CEA polypeptide can be produced using a vector in which the CAR open reading frame is followed by a T2A ribosome skip sequence and a truncated EGFR (EGFRt), which lacks the cytoplasmic signaling tail, or a truncated CD19R (also called CD19t). In this arrangement, co-expression of EGFRt or CD19t provides an inert, non-immunogenic surface marker that allows for accurate measurement of gene modified cells, and enables positive selection of gene-modified cells, as well as efficient cell tracking of the therapeutic T cells in vivo following adoptive transfer. Efficiently controlling proliferation to avoid cytokine storm and off-target toxicity is an important hurdle for the success of T cell immunotherapy. The EGFRt or the CD19t incorporated in the CEA CAR lentiviral vector can act as suicide gene to ablate the CAR+T cells in cases of treatment-related toxicity.

The CD3ζ signaling domain can be followed by a ribosomal skip sequence (e.g., LEGGGEGRGSLLTCGDVEENPGPR; SEQ ID NO: 57) and a truncated EGFR having a sequence that is at least 90%, at least 95%, at least 98% identical to or identical to: LVTSLLLCELPHPAFLLIPRKVCNGIGIGEFKDSLSINATNIKHFKNCTSISGDLHILPVA FRGDSFTHTPPLDPQELDILKTVKEITGFLLIQAWPENRTDLHAFENLEIIRGRTKQHG QFSLAVVSLNITSLGLRSLKEISDGDVIISGNKNLCYANTINWKKLFGTSGQKTKIISN RGENSCKATGQVCHALCSPEGCWGPEPRDCVSCRNVSRGRECVDKCNLLEGEPREF VENSECIQCHPECLPQAMNITCTGRGPDNCIQCAHYIDGPHCVKTCPAGVMGENNTL VWKYADAGHVCHLCHPNCTYGCTGPGLEGCPTNGPKIPSIATGMVGALLLLLVVAL GIGLFM (SEQ ID NO: 58). In some cases, the truncated EGFR has 1, 2, 3, 4 of 5 amino acid changes (preferably conservative) compared to SEQ ID NO: 58.

Alternatively the CD3ζ signaling domain can be followed by a ribosomal skip sequence (e.g., LEGGGEGRGSLLTCGDVEENPGPR; SEQ ID NO: 57) and a truncated CD19R (also called CD19t) having a sequence that is at least 90%, at least 95%, at least 98% identical to or identical to: MPPPRLLFFLLFLTPMEVRPEEPLVVKVEEGDNAVLQCLKGTSDGPTQQLTWSRESP LKPFLKLSLGLPGLGIHMRPLAIWLFIFNVSQQMGGFYLCQPGPPSEKAWQPGWTVN VEGSGELFRWNVSDLGGLGCGLKNRSSEGPSSPSGKLMSPKLYVWAKDRPEIWEGE PPCVPPRDSLNQSLSQDLTMAPGSTLWLSCGVPPDSVSRGPLSWTHVHPKGPKSLLS LELKDDRPARDMWVMETGLLLPRATAQDAGKYYCHRGNLTMSFHLEITARPVLWH WLLRTGGWKVSAVTLAYLIFCLCSLVGILHLQRALVLRRKR (SEQ ID NO: 59). In some cases, the CD19R has 1, 2, 3, 4 of 5 amino acid changes (preferably conservative) compared to SEQ ID NO: 59.

The CAR or CEA polypeptide described herein can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, overlapping PCR, primer-assisted ligation, site-directed mutagenesis, etc.) as is convenient. The resulting coding region is preferably inserted into an expression vector and used to transform a suitable expression host cell line, preferably a T lymphocyte, and most preferably an autologous T lymphocyte.

Various T cell subsets isolated from the patient can be transduced with a vector for CAR and/or ICK expression. Central memory T cells are one useful T cell subset. Central memory T cell can be isolated from peripheral blood mononuclear cells (PBMC) by selecting for CD45RO+/CD62L+ cells, using, for example, the CliniMACS® device to immunomagnetically select cells expressing the desired receptors. The cells enriched for central memory T cells can be activated with anti-CD3/CD28, transduced with, for example, a lentiviral vector that directs the expression of an anti-CEA CAR or as well as a non-immunogenic surface marker for in vivo detection, ablation, and potential ex vivo selection, and/or an ICK. The activated/genetically modified central memory T cells can be expanded in vitro with IL-2/IL-15 and then cryopreserved. Additional methods of preparing CART cells can be found in PCT/US2016/043392.

Methods for preparing useful T cell populations are described in, for example, WO 2017/015490 and WO 2018/102761. In some cases, it may be useful to use natural killer (NK) cells, e.g., allogenic NK cells derived from peripheral blood or cord blood. In other cases, NK cells can be derived from human embryonic stem cells (hESCs) or induced pluripotent stem cells (iPSCs).

In some embodiments, described herein is a composition comprising the iPSC-derived CAR T cells or CAR NK cells. In some embodiments, a composition comprising iPSC-derived CAR T cells or CAR NK cells has enhanced therapeutic properties. In some embodiments, the iPSC-derived CAR T cells or CAR NK cells demonstrate enhanced functional activity including potent cytokine production, cytotoxicity and cytostatic inhibition of tumor growth, e.g., as activity that reduces the amount of tumor load. In some embodiments, the iPSC-derived CAR T cells or CAR NK cells also express an ICK.

The CAR can be transiently expressed in a T cell population by an mRNA encoding the CAR. The mRNA can be introduced into the T cells by electroporation (Wiesinger et al. 2019 Cancers (Basel) 11:1198).

In some embodiments, a composition comprising the T cells (e.g., T cells expressing a CAR and/or an ICI( )comprise one or more of helper T cells, cytotoxic T cells, memory T cells, naive T cells, regulatory T cells, natural killer T cells, or combinations thereof.

The ICK described herein can be produced by any means known in the art, though preferably it is produced using recombinant DNA techniques. Nucleic acids encoding the anti-CEA antibody and IL-2 can be prepared and assembled into a complete coding sequence by standard techniques of molecular cloning known in the art (genomic library screening, overlapping PCR, primer-assisted ligation, site-directed mutagenesis, etc.) as is convenient. The resulting coding region is preferably inserted into an expression vector and used to transform a suitable expression host cell line, preferably CHO cells.

In some examples, separate plasmids encoding the antibody heavy chain fused to IL-2 and the antibody light chain can be used. In such instances, the plasmids can be mixed (e.g., 3:1 molar ratio of plasmid encoding the antibody heavy chain-IL-2 and plasmid encoding the antibody light chain) and transfected into the cells using any known method such as electroporation. Purification can be performed with any method suitable in the art for purifying proteins such as an anion exchange chromatography. See also Kujawski et al. Potent immunomodulatory effects of an anti-CEA-IL-2 immunocytokine on tumor therapy and effects of stereotactic radiation. 2020 OncoImmunology, 9:1, 1724052, the relevant disclosures of which are incorporated by reference herein for the purpose and subject matter referenced herein.

In some examples, the ICK comprises an amino acid sequence that is at least 90%, at least 95%, at least 98% identical to or identical to:

EVQLVESGGGLVQPGGSLRLSCAASGFNIKDTYMHWVRQAPGKGLEWVAR
IDPANGNSKYADSVKGRFTISADTSKNTAYLQMNSLRAEDTAVYYCAPFG
YYVSDYAMAYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSATATPG
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT (SEQ ID NO: 60;
IL-2 sequence is underlined)
and
(SEQ ID NO: 54)
DIQLTQSPSSLSASVGDRVTITCRAGESVDIFGVGFLHWYQQKPGKAPKL
LIYRASNLESGVPSRFSGSGSRTDFTLTISSLQPEDFATYYCQQTNEDPY
TFGQGTKVEIKRTVAAPSVFIFPPSDEQLKSGAASVVCLLNNFYPREAKV
QWKVDNALQSGNSQESVTEQDSKDSTYSLSSTLTLSKADYEKHKVYACEV
THQGLSSPVTKSFNRGEC.

IV. Treatment of Cancer

Aspects of the present disclosure provide methods for treating a subject having a cancer characterized by growth of tumor cells expressing CEA (also called a CEA-expressing cancer or a CEA-positive tumor) using any of the anti-CEA CART cells and the ICK proteins described herein. Such treatment methods can comprise a conditioning regimen (lymphodepleting treatment), which comprises administering one or more doses of a lymphodepleting agent to a subject, and a treatment regimen, which comprises administrating one or more doses of the anti-CEA CAR T cells and/or the ICK proteins to the subject.

(a) Subjects

The subject to be treated by the methods described can be a human patient having or at risk for having a CEA-expressing cancer, e.g., gastrointestinal cancer, breast cancer, lung cancer, bladder cancer, thyroid cancer, and ovarian cancer. Non-limiting examples of gastrointestinal cancers include colon cancer, gastric cancer, rectal cancer, pancreatic cancer, and combinations thereof.

A subject at risk of having a CEA-expressing cancer might show one or more symptoms of a CEA-expressing cancer, e.g., unexplained weight loss, fatigue, pain, persistent cough, lumps under the skin, or unusual bleeding. A subject at risk of having a CEA-expressing cancer might have one or more risk factors of a CEA-expressing cancer, e.g., family history of cancer, age, tobacco use, obesity, or exposure to sun or carcinogens. A subject who needs the treatment described herein can be identified by routine medical examination, e.g., laboratory tests, biopsy, magnetic resonance imaging (MRI), or ultrasound exams.

(b) Treatment Regimens

Aspects of the present disclosure provide methods of treating a CEA-expressing cancer comprising administering a lymphodepletion treatment (e.g., cyclophosphamide) in combination with anti-CEA CAR T cells and/or ICK, each of which can be administered locally or systemically. In some cases, the lymphodepletion treatment is non-myeloablative.

Any subject suitable for the treatment methods described herein can receive a lymphodepleting therapy to reduce or deplete the endogenous lymphocytes of the subject. Lymphodepletion refers to the destruction of endogenous lymphocytes and/or T cells, which is commonly used prior to immunotransplantation and immunotherapy. Lymphodepletion can be achieved by administering a lymphodepleting agent and/or irradiation (e.g., stereotactic radiation). A lymphodepleting agent can be any molecule capable of reducing, depleting, or eliminating endogenous lymphocytes and/or T cells when administered to a subject. In some to examples, the lymphodepleting agents are cytotoxic agents that specifically kill lymphocytes. Non-limiting examples of lymphodepleting agents include cyclophosphamide, fludarabine, gemcitabine, methotrexate, doxorubicin, and etopside phosphate.

Methods described herein can include a conditioning regimen comprising a single lymphodepleting agent (e.g., cyclophosphamide) or multiple lymphodepleting agents (e.g., cyclophosphamide and fludarabine). The subject to be treated by the methods described herein can receive one or more doses of the one or more lymphodepleting agents for a period suitable for reducing or depleting the endogenous lymphocytes of the subject (e.g., 1-5 days).

The subject can then be administered any of the anti-CEA CAR T cells described herein after administration of the lymphodepleting therapy as described herein. For example, the one or more lymphodepleting agents can be administered to the subject 1-5 days (e.g., 1, 2, 3, 4, or 5 days) prior to administering the anti-CEA CAR T cells.

Methods described herein can include redosing the subject with anti-CEA CAR T cells. In some examples, the subject is administered a lymphodepleting treatment prior to redosing of the anti-CAR T cells. Each dose of the anti-CEA CAR T cells can be the same or the doses can be ascending or descending.

The subject can then be administered any of the anti-CEA-IL-2 ICK described herein after administration of the anti-CEA CART cells as described herein. For example, the anti-CEA-IL-2 ICK can be administered to the subject 1-5 days (e.g., 1, 2, 3, 4, or 5 days) after administering the anti-CEA CAR T cells. Any ICK protein described herein can be administered to a subject as an ICK protein, a nucleic acid encoding an ICK protein, a cell expressing an ICK protein, or a combination thereof.

Methods described herein can include redosing the subject with the anti-CEA-IL-2 ICK. In some examples, the subject is administered 3-6 doses of the anti-CEA-IL-2 ICK, each of which is administered 1-5 days after the prior dose. Each dose of the anti-CEA-IL-2 ICK can be the same or the doses can be ascending or descending.

Methods described herein can be used in combination with another anti-cancer therapy (e.g., chemotherapy) or with another therapeutic agent that reduces side effects of the therapy described herein.

(c) Administration

An effective amount of a therapy (e.g., lymphodepleting agent, anti-CEA CAR T cells, ICK) can be administered to a subject (e.g., a human) in need of the treatment via any suitable route (e.g., administered locally or systemically to a subject). Suitable modes of administration include injection, infusion, instillation, or ingestion. Injection includes, without limitation, intravenous, intramuscular, intra-arterial, intrathecal, intraventricular, intradermal, intraperitoneal, and subcutaneous injection and infusion.

An effective amount refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of treatment, the nature of concurrent therapy, if any, the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The materials, methods, and examples are illustrative only and not intended to be limiting. All publications, patent applications, patents, sequences, database entries, and other references mentioned herein are incorporated by reference in their entirety for any and all purposes.

Other features and advantages of the described compositions and methods will be apparent from the following detailed description and figures, and from the claims.

### EXAMPLES

In order that the invention described may be more fully understood, the following examples are set forth. The examples described in this application are offered to illustrate the methods and compositions provided herein and are not to be construed in any way as limiting their scope.

### Materials and Methods

The following materials and methods were used in the Examples set forth herein.

### DNA Constructs

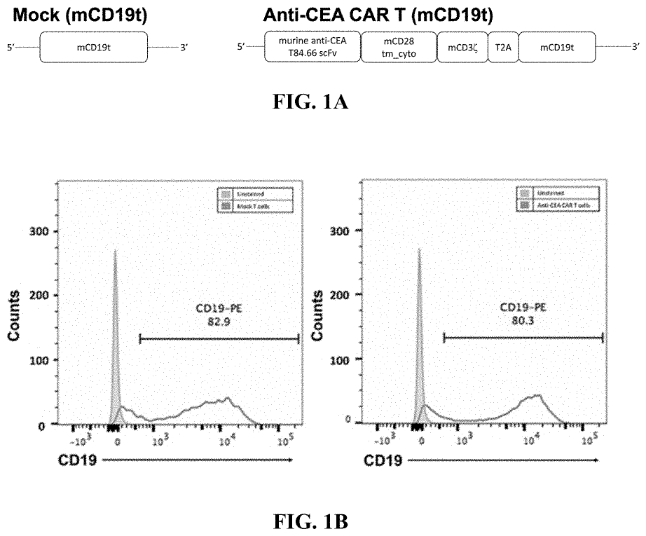

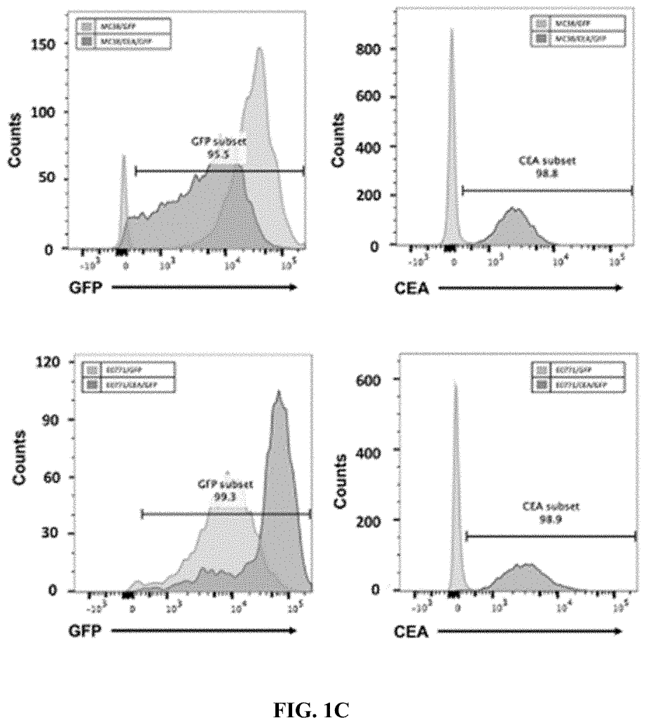

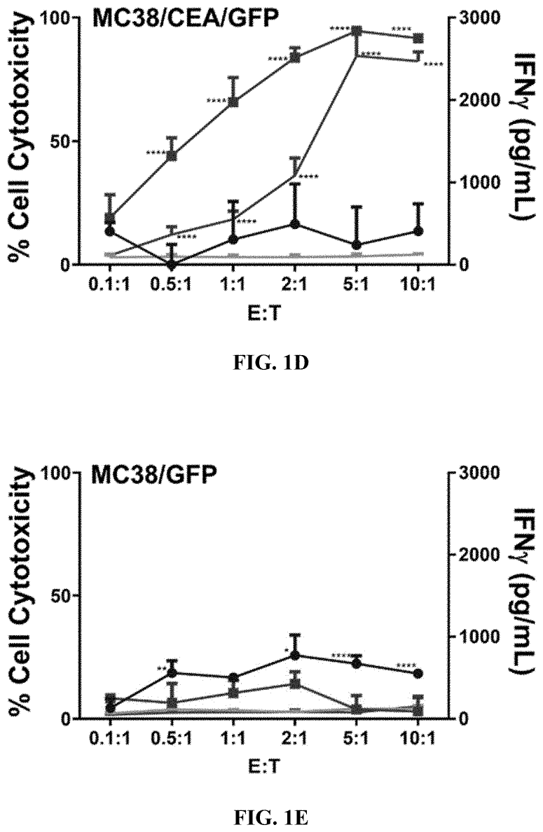

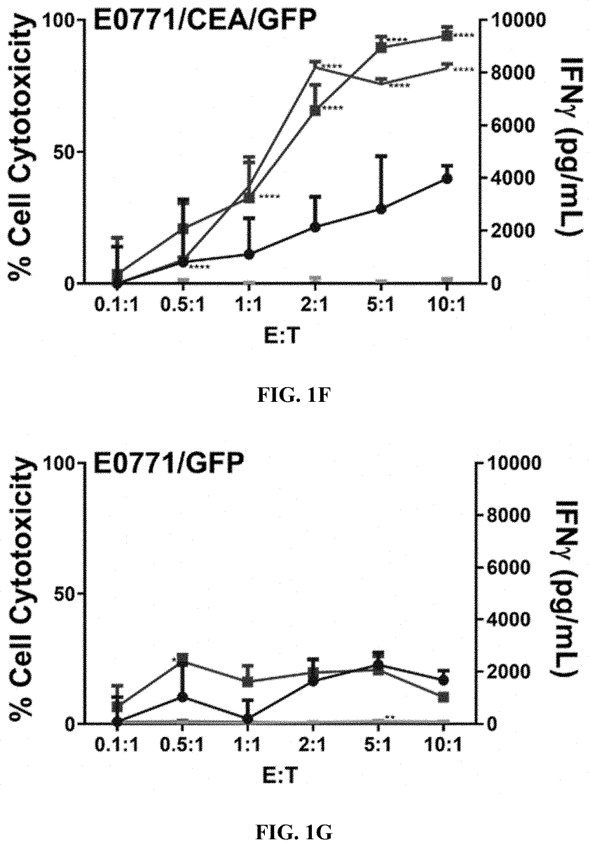

Retrovirus vector expressing GFP tag (pMIGR) was supplied as a gift from Dr. Zuoming Sun (City of Hope) (He et al. Nat Immunol, 18: 1128-38 (2017)). The GFP tag was removed from pMIGR via restriction enzyme digestion at Ncol and PacI and ligation with amino acids CCTGAA insert between the two restriction enzyme sites (pMSCV). The scFv sequence used in the CAR construct was obtained from the murine anti-CEA T84.66 monoclonal antibody (Neumaier et al. Cancer Res 50: 2128-34 (1990)). The second generation CAR construct consisted of the T84.66 scFv (VL-(GGGGS)₃-VH) fused to a 129-amino acid middle-length CH2-deleted version of the IgG4 Fc spacer (Murad et al. Front Immunol 9: 2268 (2018)) with the intracellular co-stimulatory signaling murine CD28 transmembrane and murine CD3ζ cytolytic domains. As a transduction marker, the ectodomain of mouse CD19 (mCD19t), was added downstream of the same promoter as anti-CEA CAR (Murad et al. Front Immunol 9: 2268 (2018); Brown et al. Molecular Therapy 26: 31-44 (2018); and Priceman et al. Clin Cancer Res 24: 95-105 (2018)). T2A (Donnelly et al. J Gen Virol 82: 1013-25 (2001)) was also inserted between anti-CEA CAR and mCD19t. The anti-CEA CAR construct was inserted into pMSCV vector via restriction enzyme digestion and ligation (pMSCV_anti-CEA CAR_mCD19t). As a control, mCD19t was inserted into pMSCV via PCR, restriction enzyme digestion, and ligation (pMSCV_mCD19t). After ligation, the insert was sequenced to ensure that there were no PCR-induced mutations. The constructs are shown in FIG. 1A. The immunocytokine (ICK) was produced as described by Kujawski et al. OncoImmunology, 9: 1724052 (2020).

The anti-CEA CAR mCD19t has the sequence:

(SEQ ID NO: 70)
MLLLVTSLLLCELPHPAFLLIPDIVLTQSPASLAVSLGQRATMSCRAGES
VDIFGVGFLHWYQQKPGQPPKLLIYRASNLESGIPVRFSGTGSRTDFTLI
IDPVEADDVATYYCQQTNEDPYTFGGGTKLEIKGSTSGGGSGGGSGGGGS
SEVQLQQSGAELVEPGASVKLSCTASGFNIKDTYMHWVKQRPEQGLEWIG
RIDPANGNSKYVPKFQGKATITADTSSNTAYLQLTSLTSEDTAVYYCAPF
GYYVSDYAMAYWGQGTSVTVSSTTTKPVLRTPSPVHPTGTSQPQRPEDCR
PRGSVKGTGLDFACDIYMFWALVVVAGVLFCYGLLVTVALCVIWTNSRRN
RLLQSDYMNMTPRRPGLTRKPYQPYAPARDFAAYRPGGGRAKFSRSAETA
ANLQDPNQLYNELNLGRREEYDVLEKKRARDPEMGGKQQRRRNPQEGVYN
ALQKDKMAEAYSEIGTKGERRRGKGHDGLYQGLSTATKDTYDALHMQTLA
PRLEGGGEGRGSLLTCGDVEENPGPTRMPSPLPVSFLLFLTLVGGRPQKS
LLVEVEEGGNVVLPCLPDSSPVSSEKLAWYRGNQSTPFLELSPGSPGLGL
HVGSLGILLVIVNVSDHMGGFYLCQKRPPFKDIWQPAWTVNVEDSGEMFR
WNASDVRDLDCDLRNRSSGSHRSTSGSQLYVWAKDHPKVWGTKPVCAPRG
SSLNQSLINQDLTVAPGSTLWLSCGVPPVPVAKGSISWTHVHPRRPNVSL
LSLSLGGEHPVREMWVWGSLLLLPQATALDEGTYYCLRGNLTIERHVKVI
ARSAVWLWLLRTGGWIVPVVTLVYVIFCMVSLVAFLYCQRAFILRRKR.

### Cell Lines and Culture Conditions

The Platinum-E retroviral packaging (PlatE) cell line (Cell Biolabs, RV-101) was grown in Dulbecco's Modified Eagle Medium (DMEM; Corning, 10-013-CV) supplemented with 10% fetal bovine serum (FBS), antibiotic-antimycotic solution (Coming, MT30004), 1 ug/mL puromycin (InvivoGen, ant-pr-1), and 10 μg/mL blasticidin (InvivoGen, ant-bl-1). Mouse colon adenocarcinoma cell lines with or without CEA expression (MC38/CEA or MC38, respectively) were grown in DMEM supplemented with 10% FBS, and antibiotic-antimycotic solution. GFP-expressing mouse colon adenocarcinoma cell line (MC38/GFP) and GFP-expressing mouse breast adenocarcinoma cell line (E0771/GFP) were grown in DMEM supplemented with 10% FBS, antibiotic-antimycotic solution, and 1μg/mL puromycin. GFP-expressing, CEA-positive mouse colon adenocarcinoma cell line (MC38/CEA/GFP) and GFP-expressing, CEA-positive mouse breast adenocarcinoma cell line (E0771/CEA/GFP) were grown in DMEM supplemented with 10% FBS, antibiotic-antimycotic solution, and G418 Sulfate.

### Virus Production/Transfection

PlatE cells at 80-90% confluency were transfected with either pMSCV_mCD19t or pMSCV_anti-CEA CAR_mCD19t plasmid. Prior to transfection, the medium for each plate was changed from DMEM supplemented with 10% FBS, antibiotic-antimycotic solution, 1 μg/mL puromycin, and 10 μg/mL blasticidin to DMEM supplemented with 10% FBS. Then, each plate was transfected with 14 μg of total DNA, 20 μL of PLUS solution, and 20 μL of lipofectamine LTX (Invitrogen, 15338-100) in 200 pt serum reduced OPTI-MEM (Gibco, 31985070), as per manufacturer's directions. After 24 hours from the transfection, the medium was aspirated and replaced with new DMEM supplemented with 10% FBS. After 48 and 72 hours from the transfection, the supematants were collected with 30 mL syringe (BD, 302833) and filtered through 0.45pm syringe filter (Millipore Sigma, SLHV033RS). Retrovirus in the collected supematant was either used immediately or frozen at −80° C. for later use.

### Mouse Primary CD3⁺ T Cells Isolation and Activation

Spleens from CEATg mice were separated into single cell suspension using 40 μm cell strainers (Falcon, 352340). The red blood cells were lysed using Red Blood Cell Lysis Buffer Hybri-Max (Sigma, R7757), per manufacturer's directions. The CD3⁺ cells were isolated from the primary splenocytes using EasySep Mouse T Cell Isolation Kit (StemCell, 19851), per manufacturer's directions. The CD3⁺ cells were mixed with Dynabeads Mouse T-Activator CD3/CD28 for T cell Expansion and Activation (Thermo Fisher Scientific, 11453D), per manufacturer's directions. Activation of CD3⁺ cells was performed in RPMI 1640 (Gibco, 21870076) with 10% FBS, 2mM L-glutamine, antibiotic-antimycotic solution, 10 ng/mL recombinant mouse IL7 (R&D, 407-ML), 50 IU/mL recombinant mouse IL2 (Biolegends, 575402), and 5.5 mM 0-mercaptoethanol (Gibco, 21985-023) and plated at 1×10⁶ cells/well in 24 wells plate (Coming, T-2989-24) ovemight at 37° C.

### Transduction of Activated Mouse Primary CDP T Cells

On Day 0, 24 well plates pre-coated with 20 ug/well retronectin (Takara, T100B) were incubated ovemight at 4° C., per manufacturer's directions. On Day 1, the activated CD3⁺ cells with Dynabeads Mouse T-Activator CD3/CD28 were infected in retronectin pre-coated 24 wells plate with retrovirus containing mCD19t (mock T cells) or anti-CEA CAR. Recombinant mouse IL7 (10 ng/mL), recombinant mouse IL2 (50 IU/mL), and β-mercaptoethanol (5.5mM) were added to retrovirus before transducing CD3⁺ cells. The MOI was 0.5. The transduced CD3⁺ cells were incubated overnight at 37° C. On Day 2, retrovirus was removed from each well and replaced with fresh culture media of RPMI 1640 with 10% FBS, 2mM L-glutamine, antibiotic-antimycotic solution, recombinant mouse IL7, recombinant mouse IL2, and 0-mercaptoethanol and plated at 0.5×10⁶ cells/well in 24 wells plate overnight. On Day 4, transduced CD3⁺ cells were collected. The Dynabeads Mouse T-Activator CD3/CD28 were removed from collected transduced CD3⁺ cells (StemCell, 18000). Truncated mouse CD19-positive CD3⁺ cells were positively selected using mouse CD19 Positive Selection Kit II (StemCell, 18954), per manufacturer's directions, and plated at 0.5×10⁶ cells/well in 24 wells plate overnight. On Day 5, truncated mouse CD19-positive CD3⁺ cells were collected and resuspended into 1×10⁶ cells/mL concentration for in vitro experiment and 2.5×10⁷ cells/mL concentration for in vivo experiment.

### In Vitro Killing Assay

Mock or anti-CEA CAR T cells were incubated with either MC38/GFP, MC38/CEA/GFP, E0771/GFP, or E0771/CEA/GFP cells at 0.1:1, 0.5:1, 1:1, 2:1, 5:1, or 10:1 ratio (Effectors:Targets, E:T) in RPMI 1640 medium, no phenol red (Gibco, 11835030) with 10% FBS, 2 mM L-glutamine, antibiotic-antimycotic solution, and 0-mercaptoethanol on 96 wells (Eppendorf, 951040145) overnight at 37° C. For controls, only target cells were plated. For immunocytokine (ICK) studies, ICK (12 ng/mL; equivalent to 1 ng/mL IL2) was added to media along with T cells and target cells at the beginning of 24 hours co-culture. For positive controls, 20% triton x-100 (Sigma-Aldrich, X100-100ML) in PBS was added to wells with only target cells and incubated for 30 minutes at 37° C. Supernatants were removed and collected for measurement of INFγ by ELISA. For quantitative analysis of GFP, culture media were replaced with fresh 200 μL of RPMI 1640 medium without phenol red and GFP fluorescence was read on a CLAROstar instrument. Mouse INFγ ELISA (BioLegends, 430806) was performed per manufacturer's directions. Supernatants were collected from each well of in vitro killing assay plate and diluted 1:5 for INFγ ELISA.

Cytotoxic Activity

To calculate the percentage of cell cytotoxicity, the value for 100% cell viability (valuemax) was measured by averaging the luminescence readings of target cells cultured without any T cells. The luminescence measured for each well co-cultured with effector and target cells is the experimental value (valueₑₓₚ). The background was subtracted as follows, valueₘᵢₙ from both valueₘₐₓ and valueₑₓₚ. The minimum value (valueₘᵢₙ) was calculated by taking the average of luminescence measured for target cells cultured without T cells and lysed by trypsin-EDTA. The fraction of live cell luminescence was calculated by dividing (valueₑₓₚ−valueₘᵢₙ) by (valueₘₐₓ−valueₘᵢₙ). Then, the fraction of live cell luminescence was subtracted from 1 to correspond to the luminescence lost by luciferase-expressing target cell death. The percentage of cell cytotoxicity in each well was calculated by multiplying by 100%.

$$\% cell cytotoxicity = ( 1 - \frac{v a l u {e}_{e x p} - v a l u {e}_{min}}{v a l u {e}_{max} - v a l u {e}_{min}} ) \times 100 \%$$

Flow Cytometry

For flow cytometry, cells were resuspended in PBS-2%FBS solution. Cells were incubated with appropriate staining antibody conjugated with fluorophore for 30 minutes at 4° C. in dark. T cells were stained with FITC anti-mouse CD8a (BD Biosciences, 553031), PE/Cy7 anti-mouse CD4 (Biolegend, 100422), PE anti-mouse INFγ (Biolegend, 505808), APC anti-mouse PD-1 (Biolegend, 135210), Brilliant Violet 421 anti-mouse CD127 (Biolegend, 135024), FITC anti-mouse CD19 (Biolegend, 152404), PE anti-mouse CD19 (Biolegend, 152408), PerCP/Cy5.5 anti-mouse CD4 (Biolegend, 100434), PE anti-mouse CD8b.2 (Biolegend, 140408), APC Rat Anti-Mouse CD4 (BD, 561091), FITC Rat Anti-Mouse CD8a (BD, 553030), Horizon BV711 Rat Anti-Mouse CD19 (BD, 563157), PE Rat Anti-Mouse CD4 (BD, 553049), PE-Cy7 Rat Anti-Mouse CD25 (BD, 552880), or APC Hamster Anti-Mouse CD279 (BD, 562671). T cells were stained with Anti-Mouse/Rat FoxP3 Staining Set PE (eBioscience, 72-5775-40), per manufacturer's directions. Mouse adenocarcinoma cells were stained with human CEACAM-5/CD66e APC-conjugated antibody (R&D, FAB412181A). Mouse cells were stained with Pacific Blue Annexin V (Biolegend, 640918) or Horizon Fixable Viability Stain 510 (BD, 564406) for cell viability. Flow cytometry was performed on LSRFortessa (BD) and analyzed by FlowJo software (v9 and v10). The multi-color panel was compensated with Anti-Rat and Anti-Hamster Ig κ/Negative Control Compensation Particles Set (BD, 552845) and Anti-Mouse Ig, κ/Negative Control Compensation Particles Set (BD, 552843).

Animal Model, Tumor Challenge, and Treatment

The CEA transgenic mice were previous generated by inserting a 32.6-kb fragment containing the complete human CEA gene and flanking sequences isolated from a genomic cosmid clone and used to produce transgenic C57BL/6 mice as previously described (Clarke et al. Cancer Res 58: 1469-77 (1998)). A homozygous line was established that was designated C57BL/6J-TgN(CEAGe)18FJP. Southern blot analysis showed that this line contained intact copies of the cosmid clone, with approximately 19 integrated copies at one chromosomal location. A mouse-human chimeric anti-CEA monoclonal antibody was used to examine CEA expression by immunohistochemical staining of frozen tissue sections. In the cecum and colon, approximately 20% of the luminal epithelial cells had strong cytoplasmic staining, whereas occasional glands showed intense staining. CEA was also expressed in gastric foveolar cells, whereas small intestine villi had only a few (<1%) positive cells. CEA was not found by immunohistochemistry in other tissues of the digestive tract, nor was it found in a wide range of other tissues or organs.

On Day 1, 1×10⁶ MC38/CEA or 1×10⁵ E0771/CEA cancer cells in 50-100 μL of PBS to combined with 25 μL of Matrigel (Corning, 356237) were injected s.c. or orthotopically into CEATg mouse using 28G Insulin Syringes (BD, 329461). Tumor size was measured along with each mouse's weight. Established tumors (50-75 mm³) were treated with 5×10⁶ Mock or anti-CEA CAR T cells in 200 μL PBS were injected i.v. into CEATg mice. For multiple anti-CEA CAR T cell therapy group, a second injection was performed 7 days after the first injection with third injection was given 14 days later. Depending on the study groups, lymphodepletion was induced by i.p. injection of 100 mg/kg cyclophosphamide monohydrate (Sigma, C7397-1G) one day before the start of T cell therapy. For single ICK treatment groups, 25 μg ICK was i.p. injected one day after T cell therapy. For the multiple ICK treatment groups, 25 μg ICK was i.p. injected one day after T cells and repeated every 3 days.

### Tissue Collection and Analysis

Survival studies were terminated for tumors >1500 mm³. Colon, small intestine, large intestine, stomach, liver, kidneys, spleen, lung, and heart were collected in cold PBS. Blood was collected in 0.5M EDTA (Invitrogen 15575020). Tissues were washed in PBS and frozen on dry ice using O.C.T. (FisherHealthCare, 4585) in vinyl Specimen molds (Sakura, 4557) for H&E and IHC staining. For flow cytometry analysis, small fractions of spleen were cut and meshed on 0.40 pm cell strainer (Coming, 431750) before lysing in Red Blood Cell Lysis Buffer Hybri-Max (Sigma, R7757). Blood was lysed in Red Blood Cell Lysis Buffer Hybri-Max and resuspended in PBS-2%FBS. Tumor was cut in small pieces and digested with Tumor Dissociation Kit, Mouse (MACS, 130-096-730) and gentleMACS C Tubes (MACS, 130-096-334), as per manufacturer's directions. The cells were stained with fluorescent antibodies for flow cytometry. After the surface proteins were stained, intercellular expression of FoxP3 was stained with Anti-Mouse/Rat Foxp3 Staining Set PE (eBioscience, 72-5775-40), as per manufacturer's directions. For INFγ production cells were re-stimulated using PMA (10 ng/ml; Sigma-Aldrich) and ionomycin (1 μg/ml; Sigma-Aldrich) in the presence of Brefeldin A (5 μg/ml; BioLegend, CA) in 10% FBS IMDM media for 4 hours in 37° C. Next, cells were stained for surface markers and viability marker (Zombie UV, BioLegend) and fixed and permeabilized using Foxp3 Transcription Factor Fixation/Permeabilization kit (ThermoFisher) following the manufacture's protocol and stained for intracellular INFγ (BioLegend) and analyzed by flow cytometry.

### Statistical Analysis

Cell cytotoxicity, cytokine, and mean tumor volume measurements, as well as survival over time, were analyzed via Prism software using the T-test, one-way ANOVA, or two-way ANOVA analysis.

### Example 1

### Target Specificity of Anti-CEA CAR T Cells

An all murine anti-CEA second-generation CAR consisting of a single chain variable fragment (scFv) derived from the anti-CEA T84.66 monoclonal antibody (Yazaki et al. Bioconjugate Chemistry 12: 220-8 (2001)) fused to a CH2 domain deleted IgG4 Fc murine CD28 transmembrane and CD3ζ was constructed to allow detection of anti-CEA CAR expression on mouse T cell surface (FIG. 1A). CD3⁺ T cells were transduced with mCD19t (mock T cells) or anti-CEA CAR with mCD19t (anti-CEA CART cells) and expression confirmed by detection of CD19 (FIG. 1B).

To determine the antigen-specificity of anti-CEA CAR T cells, anti-CEA CAR or mock transduced T cells were incubated with murine MC38 colon and E0771 breast adenocarcinoma cell lines transfected with CEA and GFP in increasing Effector:Target (E:T) ratios with GFP only as a positive control. GFP and CEA expression were confirmed by flow cytometric detection (FIG. 1C). Specific lysis of CEA⁺ vs CEA⁻ target cells with anti-CEA CAR T cells was demonstrated, while mock transduced T cells were ineffective in killing targets (FIGS. 1D-1G). For both CEA⁺ target cells, only anti-CEA CAR T cells showed a dose response of increasing levels of INFγ with increasing E:T ratios (FIG. 1D and FIG. 1F). Together, these data confirmed that anti-CEA CAR T cells specifically target CEA-expressing cells via antigen recognition and T cell activation.

### Example 2

### Delay of CEA⁺ Colon Adenocarcinoma Tumor Growth by Anti-CEA CAR T Therapy

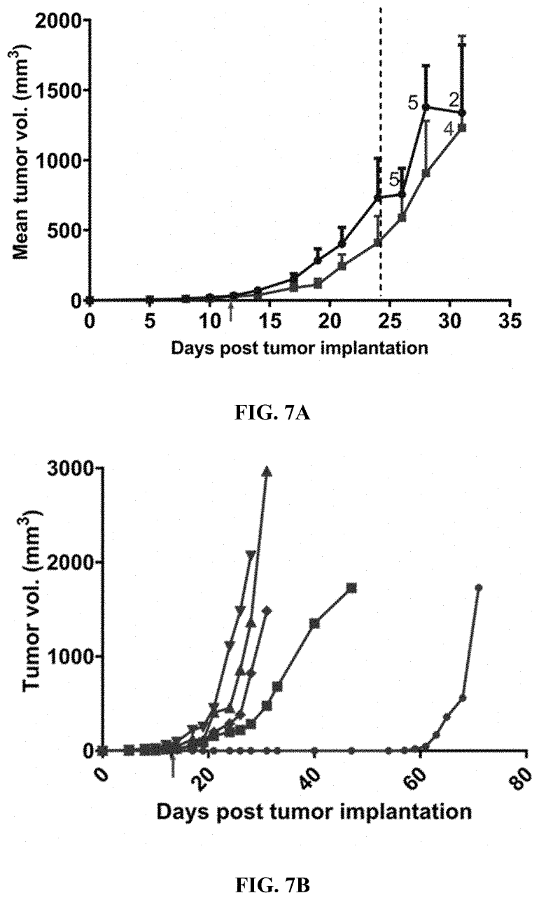

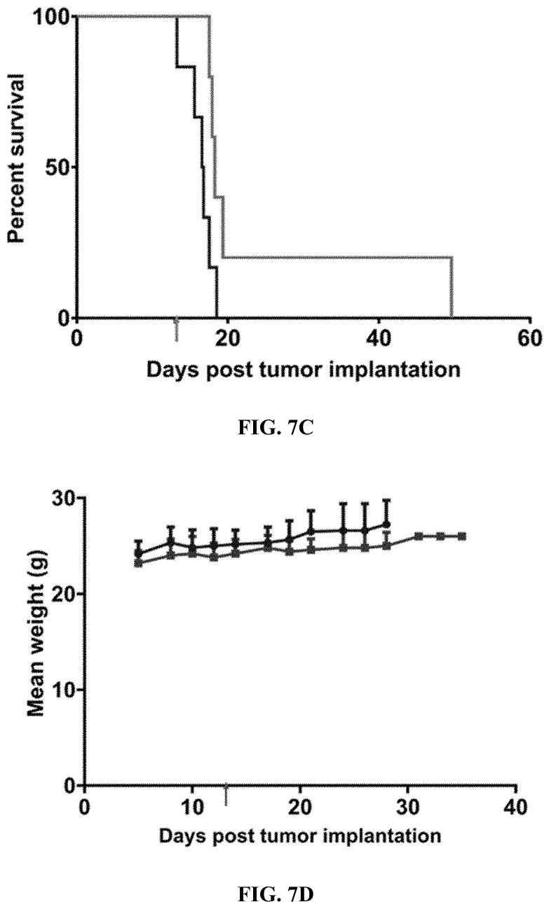

To evaluate the therapeutic potential of anti-CEA CAR T cells, MC38/CEA colon tumors were implanted in immunocompetent CEA transgenic (CEATg) mice and treated with a single i.v. injection of either anti-CEA CAR or mock transduced T cells. There was a significant reduction in tumor growth in anti-CEA CAR T cell treated mice compared to mock transduced T cell treated mice up to day 24 (FIG. 7A). After Day 24, a number of mice treated with mock transduced T cells had to be euthanized due to large tumor size. One out of 5 mice treated with anti-CEA CAR T cells exhibited tumor regression out to day 54 with subsequent tumor regrowth followed out to day 71 post tumor implantation (FIG. 7B). The median overall survival for mice treated with anti-CEA CAR T cells was statistically significant compared to mice treated with Mock T cells (p<0.02, FIG. 7C). No whole-body toxicity was observed as measured by mouse weight (FIG. 7D), as well as absence of diarrhea or loss of physical mobility. These data indicate that systemic delivery of anti-CEA CAR T cells in this model resulted in modest delays in CEA⁺ tumor growth without severe toxicity in immune competent CEATg mice. This is in agreement with other studies that have shown that CAR T cell therapy alone was not sufficient to eradicate solid tumors (Crouch et al. PLoS One 12: e0181086 (2017)).

### Example 3

### Immunodepletion Improves the Efficacy of Anti-CEA CAR T Cell Therapy

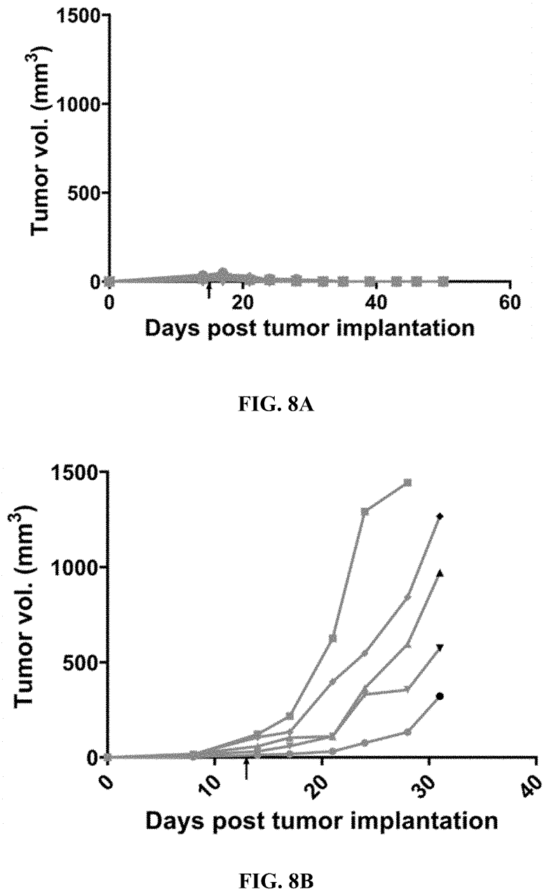

Since the administration of exogenous T cells in immunocompetent mice leads to homeostatic reduction of T cells, T cell depletion by short acting agents such as cyclophosphoamide (CY) can improve CAR T therapy. CY, an alkylating agent, is a commonly used chemotherapeutic agent that selectively depletes immunosuppressive cells, such as regulatory T cells, to increase antitumor activity. While CY treatment alone is also not sufficient to eradicate most solid tumors, it was quite effective in the treatment of MC38 tumors as shown by Myers et. al. Oncotarget 8: 5426-38 (2017). Although MC38/CEA tumors were treated with the same doses of CY as MC38 tumors, MC38/CEA tumors were resistant to CY treatment (FIG. 8A and FIG. 8B), in agreement with the chemoresistant effect of CEA expression in colon cancers (Eftekhar & Naghibalhossaini Mol Biol Rep 41: 459-66 (2014)). For this reason the combination therapy was performed only on mice bearing MC38/CEA tumors.

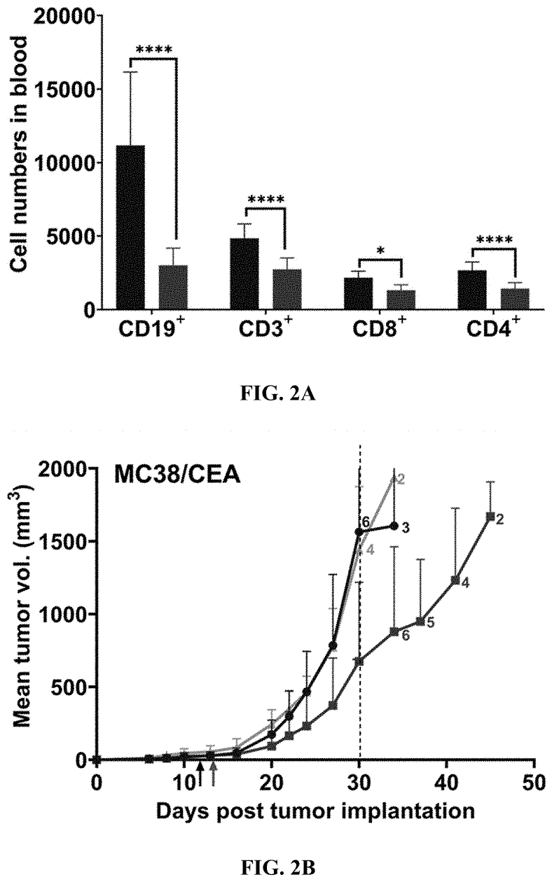

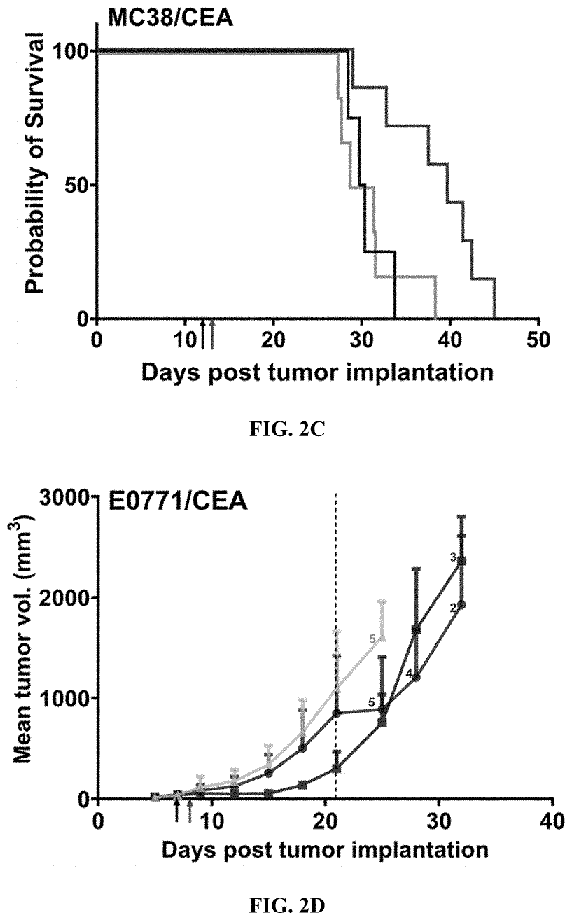

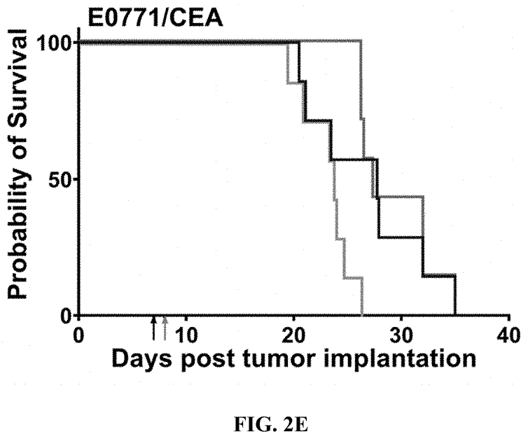

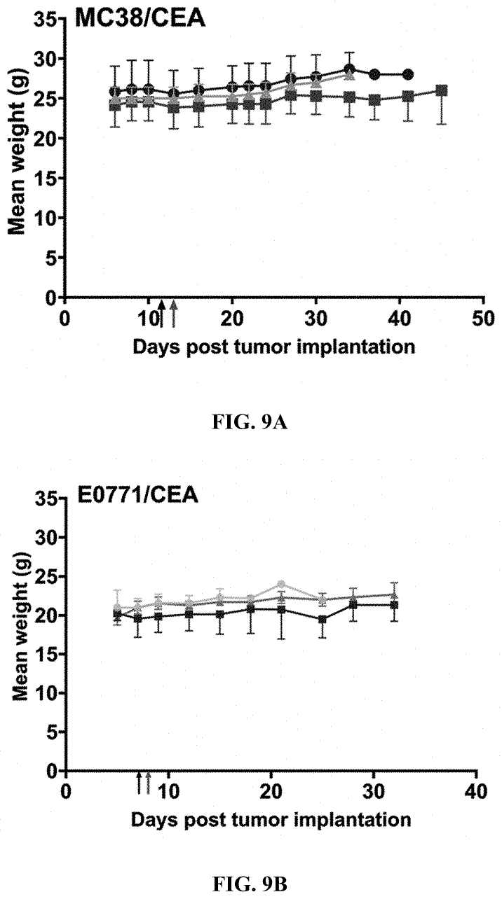

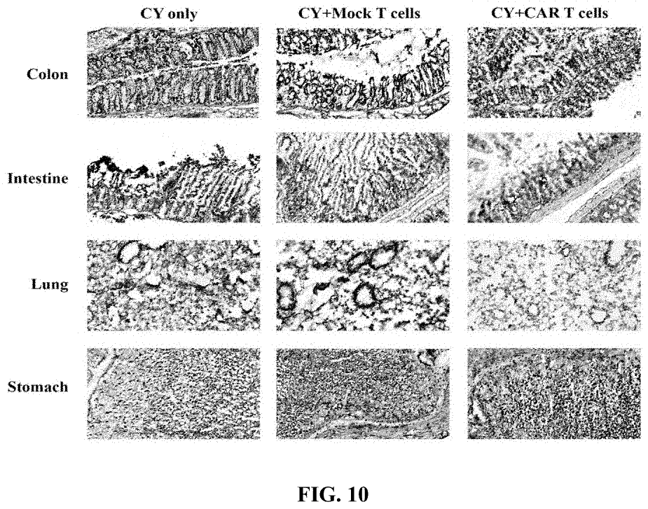

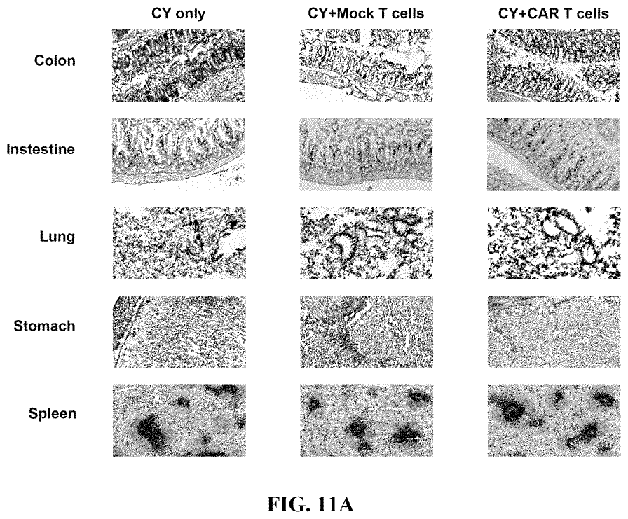

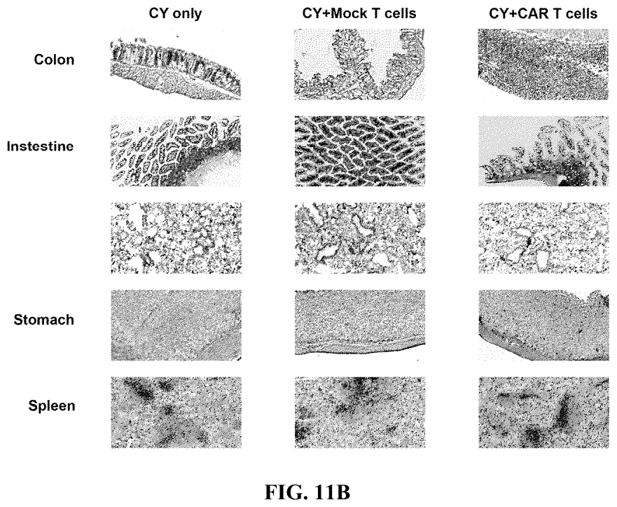

CY i.p. injected into CEA⁺ tumor-bearing, immunocompetent CEATg mice 24 hours prior to CAR T cell therapy depleted both B cells and T cells, 73% and 43%, respectively (FIG. 2A). CY treatment combined with anti-CEA CAR T cell therapy delayed MC38/CEA tumor growth by 30 days compared to 24 days for anti-CEA CAR T cell therapy alone (FIG. 2B and FIG. 7A). The median overall survival in mice treated with CY plus anti-CEA CAR T cells had a statistically significant increase to 40 days compared to 30 days for mice treated with CY and mock T cells (p<0.01) (FIG. 2C). The median survival in mice treated with CY alone was also 30 days (FIG. 2C). No whole-body toxicity was observed as measured by a decrease in mouse weight (FIGS. 9A-9B), as well as no diarrhea or loss of physical mobility. There were no morphology changes in CEA⁺ organs that were collected three days post T cell therapy and stained for human CEA (FIG. 10), confirming the expression of CEA in the transgenic mice. Staining of the CEA⁺ tissues—collected 3 days post-T cell therapy (FIG. 11A) vs the terminal timepoint of 1500 mm³ tumor size (FIG. 11B)—for murine CD3ζ to revealed the presence of murine T cells in these tissues. In addition, there was no evidence of morphology changes in CEA⁺ organs at either the early or later timepoints, indicating lack of inflammatory tissue damage or presence of non-cytotoxic tissue infiltrating T cells.

To evaluate the efficacy of combined CY plus anti-CEA CAR T cell therapy in a more physiologically relevant tumor, orthotopic E0771/CEA breast adenocarcinoma tumor-bearing CEATg mice were treated with the combined treatments. The combined treatments delayed tumor growth by 21 days (FIG. 2D) compared to 30 days for the MC38/CEA tumors (FIG. 2B). Mice bearing E0771/CEA tumors (N=7) treated with CY and anti-CEA CAR T cells survived for 26 days post tumor implantation, while mice treated with CY alone or with CY plus Mock transduced T cells had a median survival of 19 days and 20 days, respectively (FIG. 2E). No whole-body toxicity was observed as measured by a decrease in mouse weight (FIGS. 9A-9B), as well as no diarrhea or loss of physical mobility. These data suggest that CY improves antitumor activity of systemically delivered anti-CEA CAR T cells against both subcutaneous and orthotopic adenocarcinoma tumors without inducing off-target toxicity in CEATg mice.

### Example 4

### Immune Cell Infiltrate Phenotype Analysis

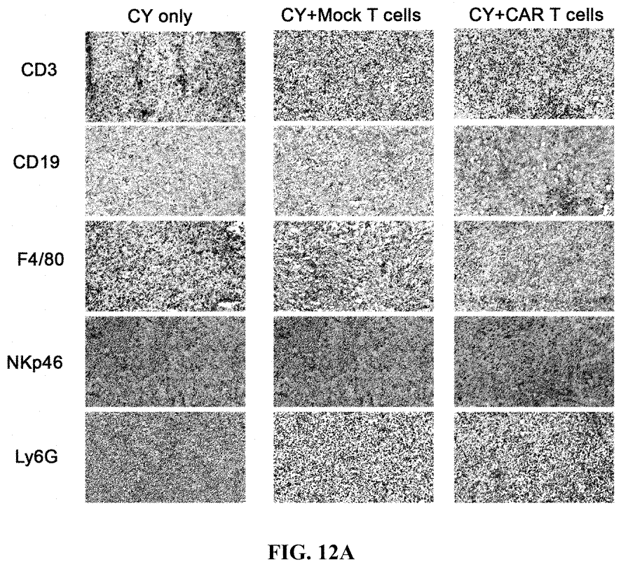

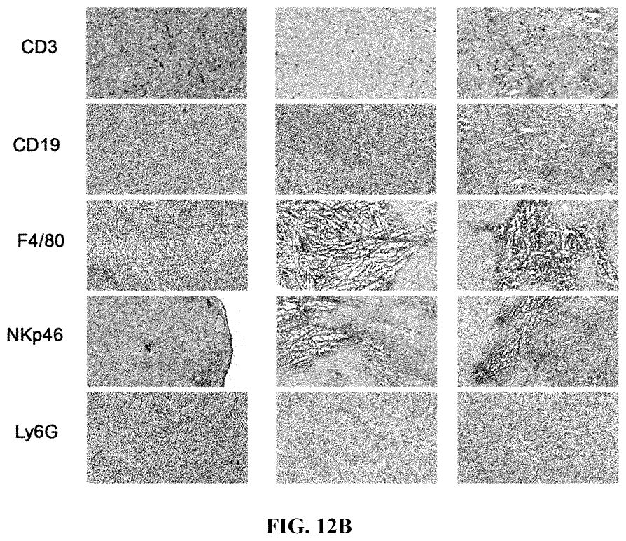

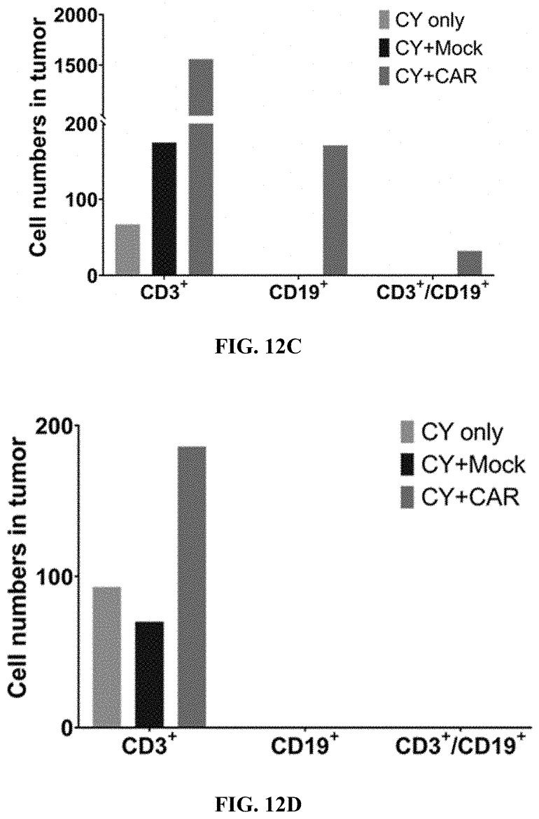

To determine the effects of CY, MC38/CEA tumors were collected three days after CY plus either mock transduced T cell or anti-CEA CAR T cell therapy or at the termination of a study group (maximum tumor size of 1500 mm³). The collected tumors were sectioned and analyzed by immunohistochemistry to detect immune cell infiltration into tumor. Three days after CY and T cell treatments, high levels of CD3⁺ T cells, F4/80⁺ macrophages, and NKp46⁺ natural killer cells were found in tumors of all treatment groups (FIG. 12A). The highest levels of CD3⁺ T cells and F4/80⁺ macrophages were found in the tumors treated with CY plus anti-CEA CAR T cells (FIG. 12A). When tumors grew to the maximum size of 1500 mm³, there were much lower levels of CD3⁺ T cells, F4/80⁺ macrophages, and NKp46⁺ natural killer cells were found in tumors of all treatment groups (FIG. 12B). Interestingly, F4/80⁺ macrophages and NKp46⁺ natural killer cells detected at the termination timepoint were not evenly spread throughout the tumor compared to the 3 day post-therapy timepoint (FIG. 12A) but mostly found at the tumor edges or in streaks (FIG. 12B). Low levels of CD19⁺ B cells and few CD3⁺/CD19⁺ anti-CEA CAR T cells were found only in the 3 day post-therapy tumor treated with CY plus anti-CEA CAR T cells (FIG. 12C), evidence for CAR T cells that trafficked into the tumor. Once the tumor grew to 1500 mm³, no CD19⁺ B cells or CD3⁺/CD19⁺ anti-CEA CART cells were found in any of the tumors (FIG. 12D). There was evidence of Ly6G⁺ neutrophils only in 3 days post-therapy tumors treated with CY plus anti-CEA CAR T cells and none in tumors treated with any other treatments or in tumors at the terminal timepoint (FIG. 12A and FIG. 12B).

### Example 5

### Persistence of CEA Expression After CY and T Cell Therapy

In hematological malignancies, antigen loss after CAR T cell therapy has been observed and correlated with relapse. As result, CAR T cell therapy in these patients required therapy with a second tumor-associated antigen. To determine if CEA loss occurred in the CAR T therapy described herein, MC38/CEA tumors were collected from mice with or without CY and/or CAR T cell therapy and then immunostained for CEA. CEA expression persisted in all MC38/CEA tumors after all treatments (FIG. 3). These data indicate that anti-CEA CAR T cell therapy can be repeatedly used to treat CEA⁺ tumors without cancer escape via antigen loss.

### Example 6

### Pre-Treatment with CY Plus Multiple Anti-CEA CAR T Cell Therapy Improves Outcome

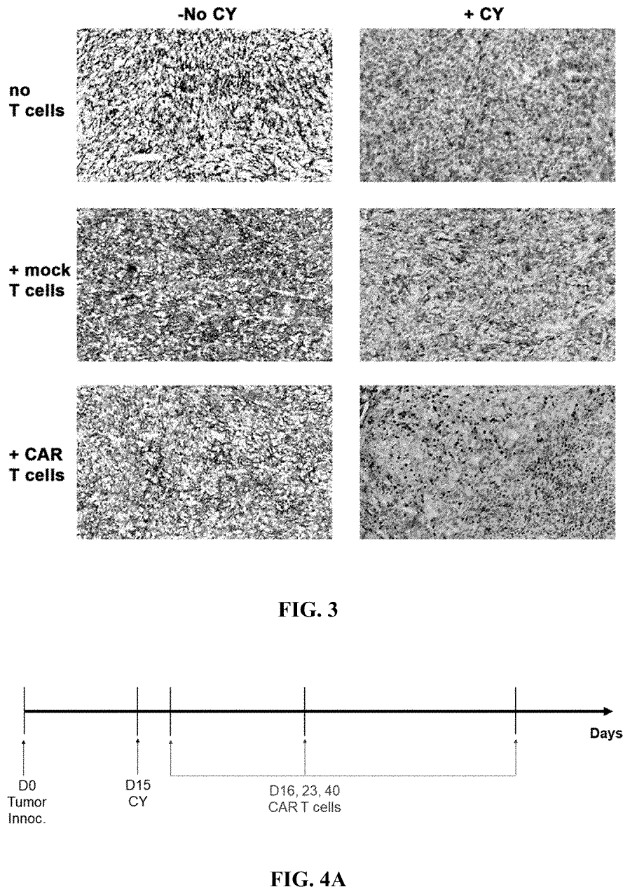

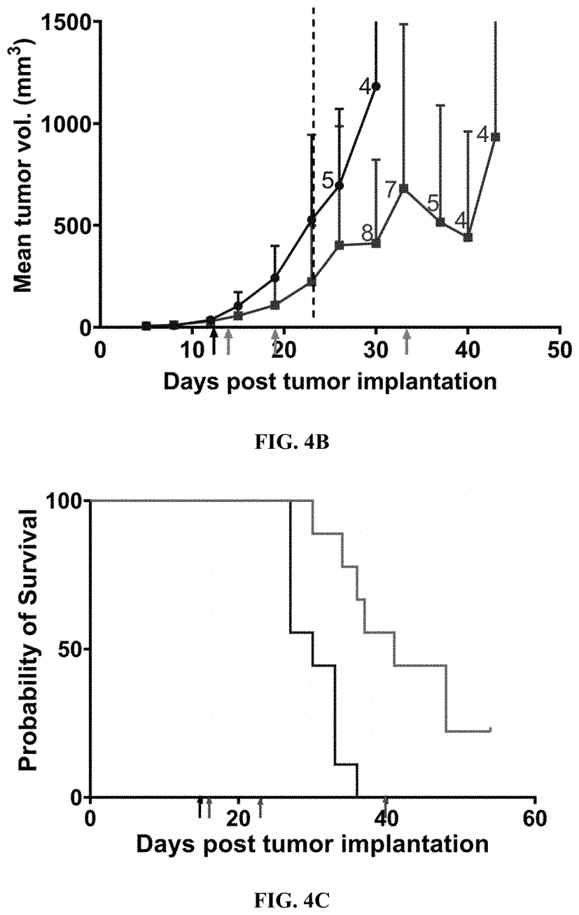

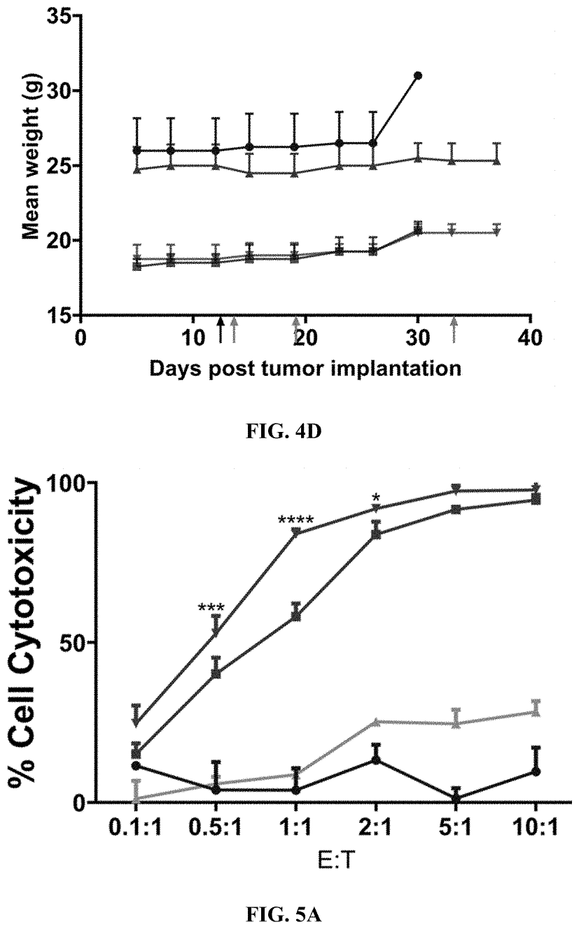

To prolong the antitumor activity of anti-CEA CAR T cell therapy, anti-CEA CAR T cell therapy was administered weekly for 3 weeks following CY treatment (FIG. 4A). Two of 9 mice had complete eradication of MC38/CEA tumors after CY plus multiple anti-CEA CAR T cell therapies (FIG. 4B). The median overall survival in mice treated with CY and multiple anti-CEA CART cell therapies was 41 days vs. 30 days in mice treated with CY plus mock transduced T cells (p<0.001, FIG. 4C). There was no decrease in mouse weight (FIG. 4D), or evidence of diarrhea and loss of physical mobility. These data suggest that, despite comparable overall survival between single and multiple anti-CEA CAR T cell therapies following CY treatment, multiple anti-CEA CAR T cell therapies may increase the incidence of tumor eradication without off-target toxicity in the CEATg mouse model.

### Example 7

### Immunocytokine Anti-CEA-IL2 Increases the Cytotoxic Activity of Anti-CEA CAR T Cells

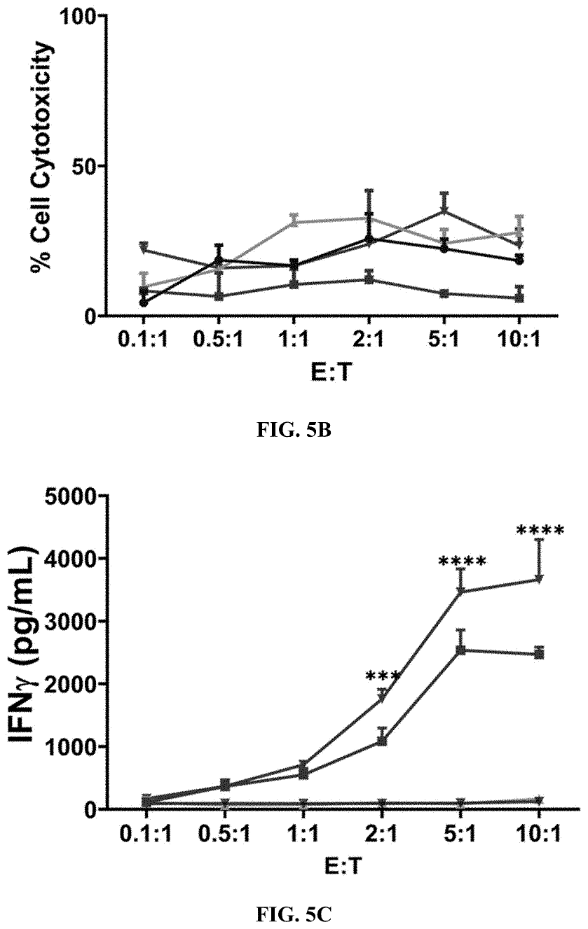

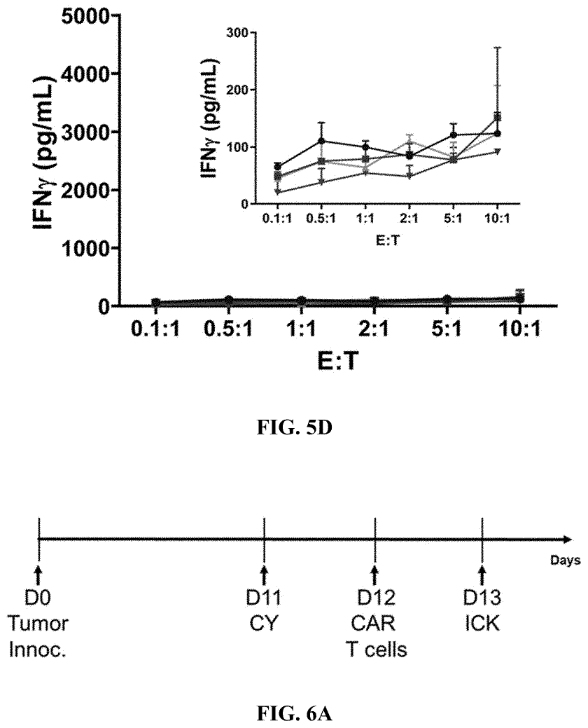

Although several anti-CEA CAR T cell clinical trials have added systemic IL2 to therapy to improve efficacy (NCT01373047; NCT02850536; NCT02416466; NCT03818165), patients were taken off IL2 treatment due to systemic IL2-related toxicity (Katz et al. Clin Cancer Res 21: 3149-59 (2015)). It has been shown that systemic IL2 toxicity can be reduced by the use of targeted immunocytokines (Kujawski et al. Oncolmmunology, 9: 1724052 (2020); Klein et al. OncoImmunology 6: e1277306 (2017); Ribba et al. Clin Cancer Res 24: 3325-33 (2018); van Brummelen et al. Oncotarget 9: 24737-49 (2018)). Specifically, combined stereotactic radiation therapy (SRT) with anti-CEA-IL2 in the MC38/CEA and E0771/CEA tumor models in CEATg mice was found to not only stimulate a T cell mediated antitumor response but also to lower regulatory T cell infiltration and stimulate a memory antitumor effect (Kujawski et al. Oncolmmunology, 9: 1724052 (2020)). To determine if the addition of ICK to anti-CEA CAR T cell therapy would also improve the antitumor response, anti-CEA CAR or mock transduced T cells were co-cultured with MC38/GFP vs MC38/CEA/GFP cells in in the presence or absence of anti-CEA-IL-2 ICK. The anti-CEA CAR T cells with ICK exhibited a dose dependent cytotoxic response to CEA⁺ vs CEA⁻ cells and secreted more INFγ than anti-CEA CAR T cells alone or mock transduced T cells (FIGS. 5A-5D). As expected, ICK co-cultured with mock transduced T cells also increased their cytotoxicity against target cells, but INFγ secretion was not increased (FIGS. 5C-5D). These data demonstrate that ICK increases the cytolytic activity of anti-CEA CAR T cells via enhancing antigen-specific T cell activation.

### Example 8

### ICK Treatment Improves Efficacy of Anti-CEA CAR T Cell Therapy against Subcutaneous and Orthotopic CEA⁺ Adenocarcinoma Tumors in CEATg Mice

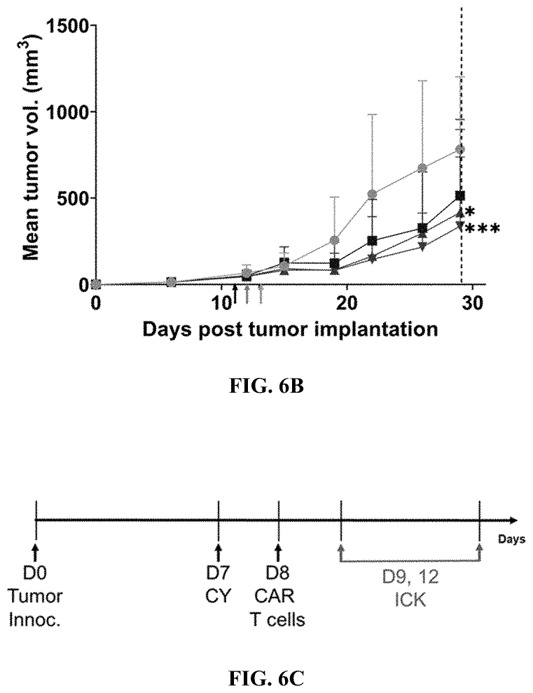

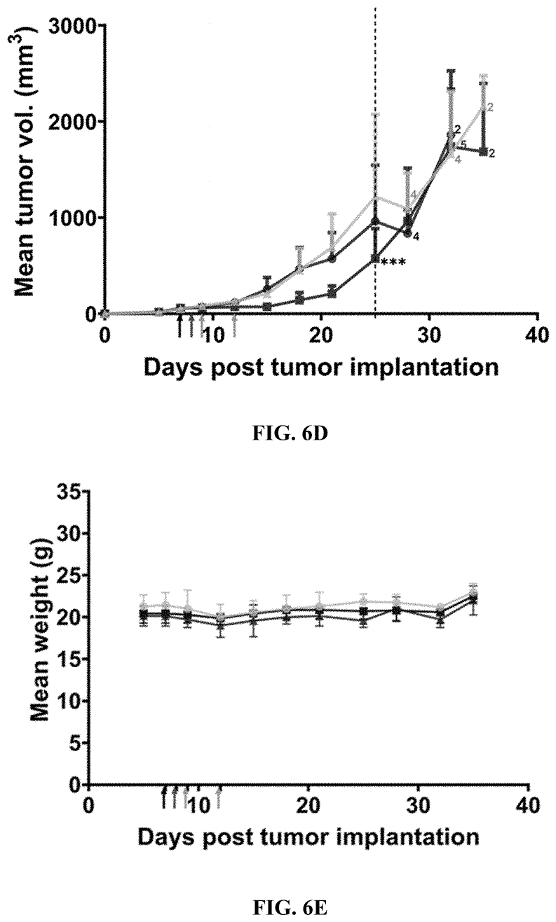

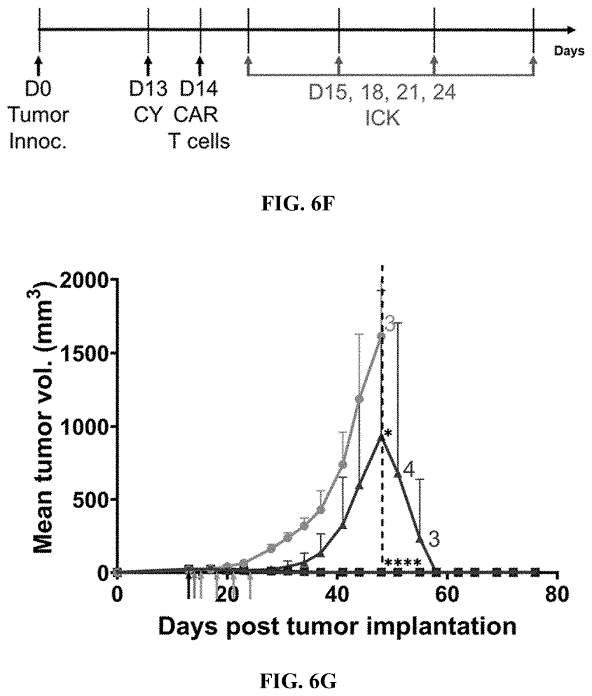

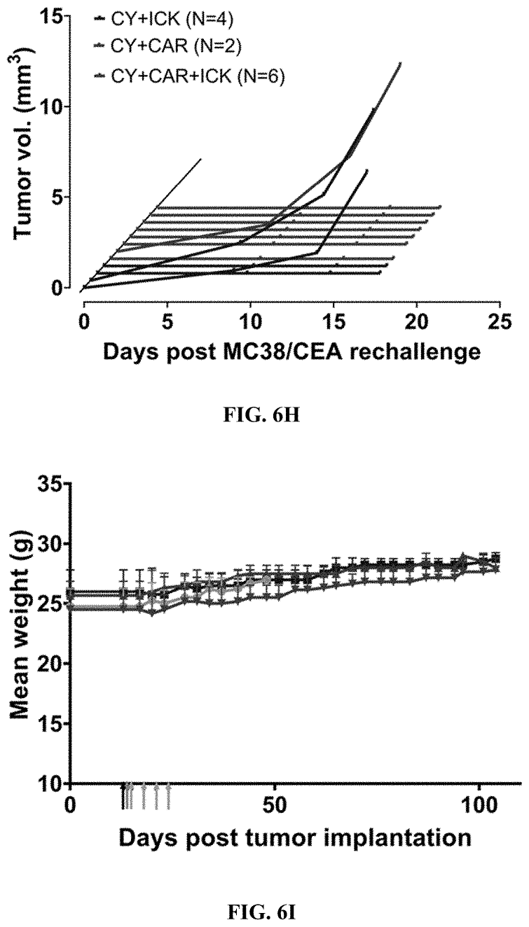

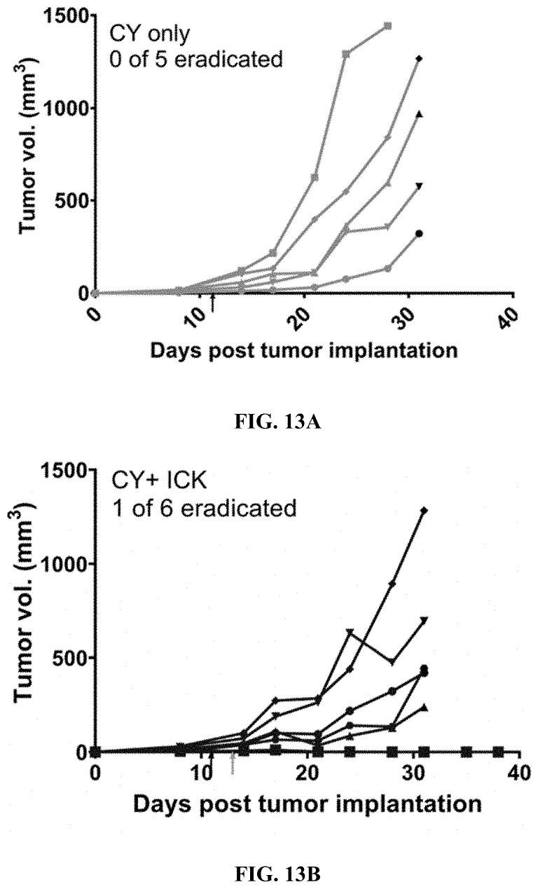

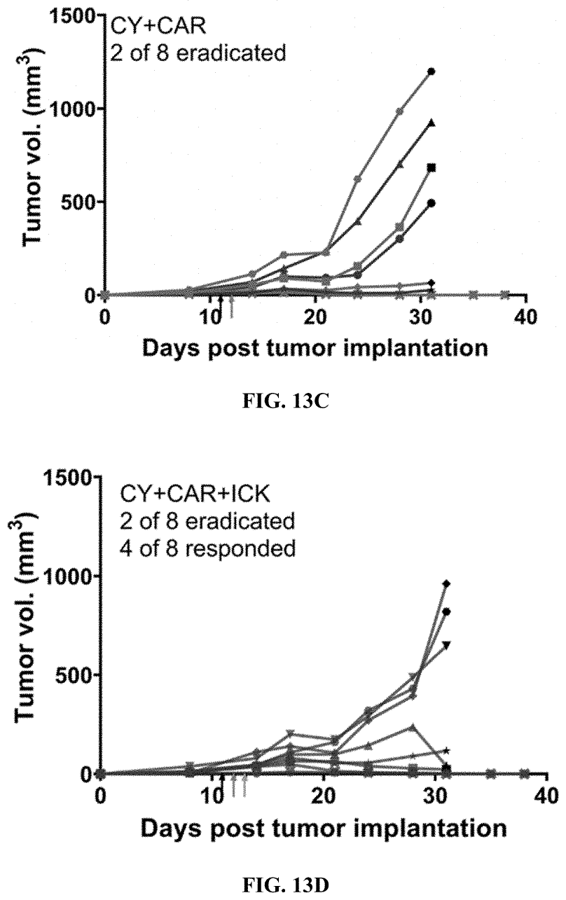

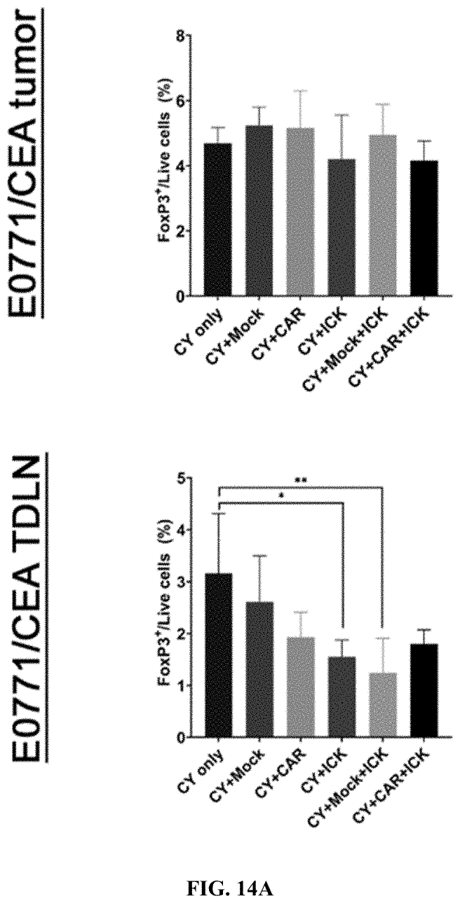

To evaluate the effects of ICK on the efficacy of anti-CEA CAR T cells in vivo, MC38/CEA tumor-bearing CEATg mice were given an i.p. injection of ICK one day following the start of CY and anti-CEA CAR T cell therapy (FIG. 6A). Combined treatment of CY and CAR T cells plus ICK therapy resulted in MC38/CEA tumor regression in 4/8 CEATg mice compared to 2/8 mice treated with CY and CAR T cells without ICK (FIG. 6B and FIGS. 13A-13D).

Since a single ICK treatment added to CY plus anti-CEA CAR T cell therapy showed potential in improving the delay of tumor growth, two ICK injections were added to the therapy of the more aggressive E0771/CEA tumors (FIG. 6C). Two ICK treatments, three days apart, added to the CY plus anti-CEA CART cell therapy delayed E0771/CEA tumor growth to 25 days compared to 21 days for mice treated with CY and anti-CEA CAR T cells alone (FIG. 6D and FIG. 2D). As before, there was no decrease in mouse weights, or evidence of diarrhea or loss of physical mobility (FIG. 6E). These data indicate that ICK enhances antitumor activity of anti-CEA CART cells against both MC38/CEA and E0771/CEA tumors without causing off-target toxicity in CEATg mice.

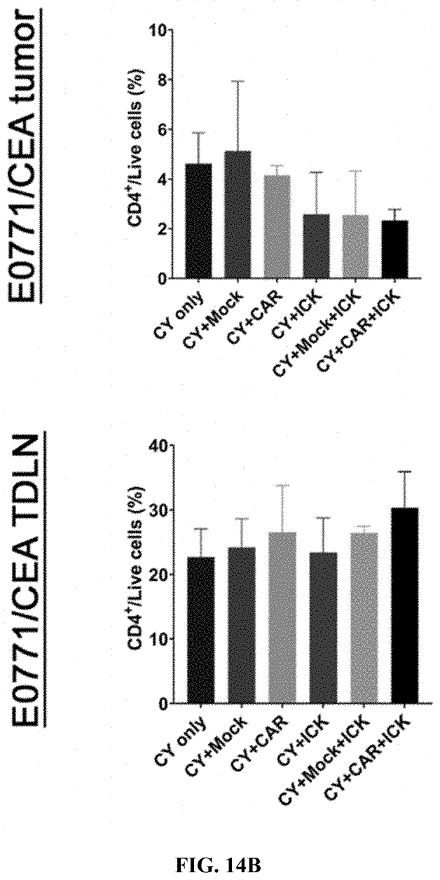

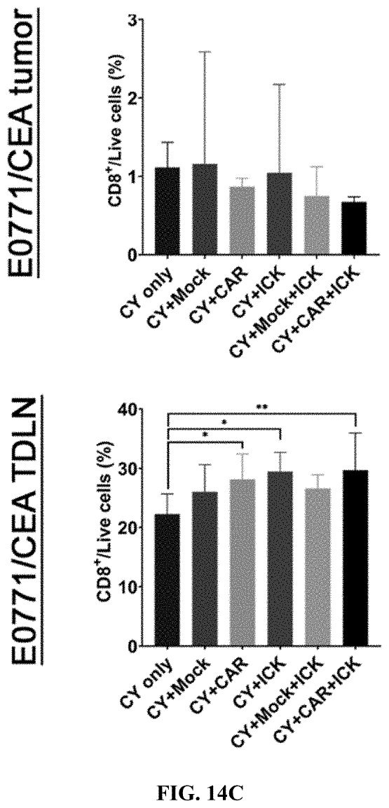

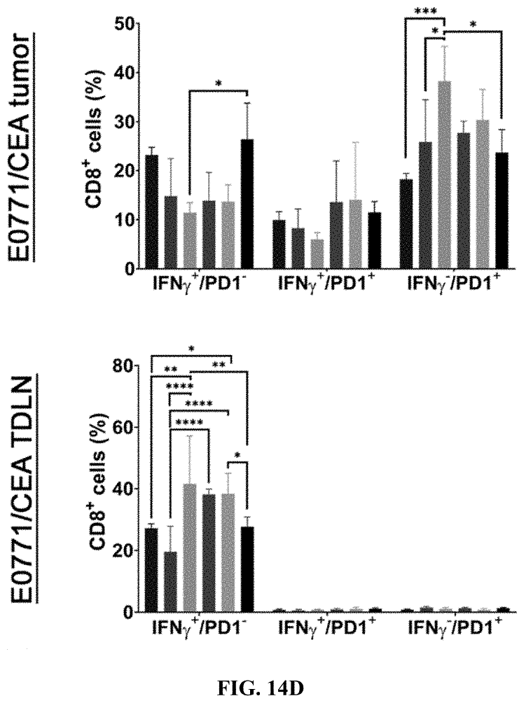

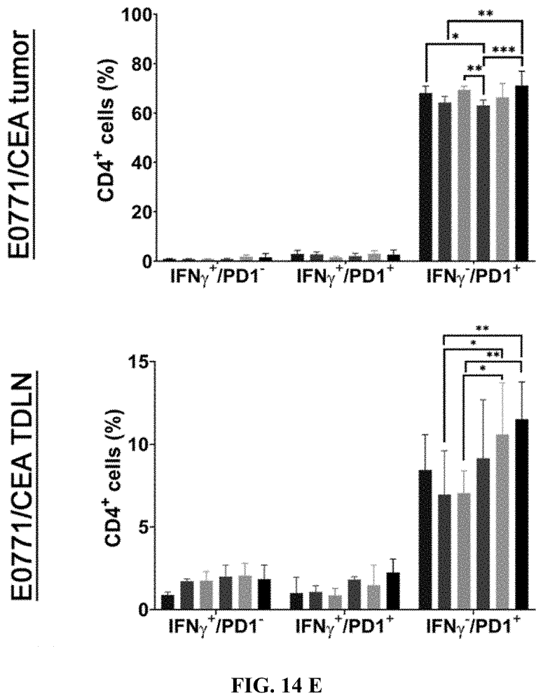

Lymphocytes in the tumors and tumor-draining lymph nodes (TDLNs) of the orthotopic E0771/CEA tumor model were analyzed by flow cytometry. Tissues were analyzed at the experimental end point of maximum allowed tumor volumes in which tumors relapsed. Nevertheless, mice treated with ICK had a significantly lower levels of tumor-infiltrating FoxP3⁺ regulatory T cells (Treg) than mice not treated with ICK, but the percent of FoxP3⁺ Treg cells was not affected by ICK in the TDLNs (FIG. 14A). In all treatment groups, more CD4⁺ and CD8⁺ T cells were found in the TDLNs than in the tumors (FIGS. 14B-14C). Tumor-infiltrating CD8⁺ T cells were IFNγ⁺/PD1⁻, IFNγ⁺/PD1⁺, and IFNγ⁻/PD1⁺ but CD8⁺ T cells in TDLNs were predominantly IFNγ⁺/PD1⁻ (FIG. 14D). CD4⁺ T cells in the tumors and in the TDLNs were mostly IFNVPD1⁺ (FIG. 14E). These data indicate that CY plus anti-CEA CART cells combined with ICK predominantly recruit IFNγ⁺/PD1⁻ CD8⁺ T cells from TDLNs into tumors and that ICK promotes a pro-inflammatory tumor microenvironment.

When the number of ICK treatments post CY plus CART cell therapy were increased to four times, three days apart, in the MC38/CEA tumor model (FIG. 6F), tumor eradication was obtained in 6/6 treated mice compared to 2/6 in the CY plus anti-CEA CAR T cell group (FIG. 6G). However, 4/4 mice treated with CY plus ICK also exhibited complete tumor eradication (FIG. 6G), suggesting that CY plus 4 treatments of ICK alone was sufficient. To determine if CAR T therapy added benefit, mice with tumor regression were re-challenged with fresh tumor after 45 tumor-free days. Within seven days after the re-challenge, MC38/CEA tumors recurred in 0/6 of the CY plus anti-CEA CAR T cells plus ICK group, 2/4 of the CY plus ICK group, and 1/2 of the CY plus anti-CEA CART cells group (FIG. 6H). As before, there was no decrease in mouse weight, no evidence of diarrhea, and no loss of physical mobility (FIG. 61). These data indicate that four ICK treatments following CY plus anti-CEA CAR T cell treatment was sufficient to not only eradicate MC38/CEA tumors but also to establish tumor immunity.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a subject having a cancer characterized by growth of tumor cells expressing carcinoembryonic antigen (CEA), the method comprising:
administering to the subject a population of T cells expressing a chimeric antigen receptor (CAR) that binds CEA and an anti-CEA-IL-2 immunocytokine (ICK),
wherein the CAR that binds CEA comprises:
a single-chain variable fragment (scFv) that binds CEA and comprises a light chain complementary determining region 1 (LC CDR1) of SEQ ID NO: 7, a light chain complementary determining region 2 (LC CDR2) of SEQ ID NO: 8, and a light chain complementary determining region 3 (LC CDR3) of SEQ ID NO: 9, and a heavy chain complementary determining region 1 (HC CDR1) of SEQ ID NO: 11, a heavy chain complementary determining region 2 (HC CDR2) of SEQ ID NO: 12 or SEQ ID NO: 15, and a heavy chain complementary determining region 3 (HC CDR3) of SEQ ID NO: 13;
a spacer domain;
a transmembrane domain;
a co-stimulatory domain, and a CD3ζ cytoplasmic signaling domain; and
wherein the anti-CEA-IL-2 ICK comprises:
a heavy chain variable domain (VII) comprising a HC CDR1 of SEQ ID NO: 11, a HC CDR2 of SEQ ID NO: 12 or SEQ ID NO: 15, and a HC CDR3 of SEQ ID NO: 13;
a light chain variable domain (V_{L}) comprising a LC CDR1 of SEQ ID NO: 7, a LC CDR2 of SEQ ID NO: 8, and a LC CDR3 of SEQ ID NO: 9; and IL-2.

2. The method of claim 1, wherein the population of T cells is administered 1 to 3 days prior to administering the anti-CEA-IL-2 ICK.

3. The method of claim 1, further comprising administering at least one additional dose of the anti-CEA-IL-2 ICK.

4. The method of claim 3, wherein the at least one additional dose of the anti-CEA-IL-2 ICK comprises 3 to 6 doses, each of which is administered 1 to 5 days after the prior dose.

5. The method of claim 1, further comprising administering to the subject a lymphodepleting agent.

6. The method of claim 5, wherein the lymphodepleting agent is administered 1 to 3 days prior to administering the population of T cells.

7. The method of claim 5, wherein the lymphodepleting agent is cyclophosphoamide (CY).

8. The method of claim 1, further comprising treating the subject with an additional anti-cancer therapy.

9. The method of claim 8, wherein the additional anti-cancer therapy is stereotactic radiation therapy (SRT).

10. The method of claim 1, wherein the scFv or the Fab of the CAR comprises a light chain complementary determining region 1 (LC CDR1) of SEQ ID NO: 7, a light chain complementary determining region 2 (LC CDR2) of SEQ ID NO: 8, and a light chain complementary determining region 3 (LC CDR3) of SEQ ID NO: 9, and a heavy chain complementary determining region 1 (HC CDR1) of SEQ ID NO: 11, a heavy chain complementary determining region 2 (HC CDR2) of SEQ ID NO: 12, and a heavy chain complementary determining region 3 (HC CDR3) of SEQ ID NO: 13.

11. The method of claim 10, wherein the scFv of the CAR comprises a V_{L} comprising SEQ ID NO: 6, and a V_{H} comprising SEQ ID NO: 10 or SEQ ID NO: 14.

12. The method of claim 11, wherein the scFv of the CAR comprises SEQ ID NO: 5.

13. The method of claim 10, wherein the Fab comprises a V_{L} comprising SEQ ID NO: 61, a light chain constant region (C_{L}) comprising SEQ ID NO: 62, a V_{H} comprising SEQ ID NO: 63, and a heavy chain constant region 1 (C_{H}1) comprising SEQ ID NO: 64.

14. The method of claim 13, wherein the Fab comprises SEQ ID NO: 65.

15. The method of claim 1, wherein the spacer domain comprises an a3 spacer domain, a linker domain, an IgG4 hinge or variant thereof, a CD28 hinge or a variant thereof, a CD8 hinge, or a combination thereof.

16. The method of claim 15, wherein the spacer domain comprises any one of SEQ ID NOs: 28-38.

17. The method of claim 1, wherein the transmembrane domain comprises a CD3ζ transmembrane domain, a CD8 transmembrane domain, a CD28 transmembrane domain, a CD4 transmembrane domain, a 41BB transmembrane domain, a NKG2D transmembrane domain, or a combination thereof.

18. The method of claim 17, wherein the transmembrane domain comprises any one of SEQ ID NOs: 18-27.

19. The method of claim 1, wherein the co-stimulatory domain is selected from the group consisting of a CD3ζ co-stimulatory domain or variant thereof, a CD28 co-stimulatory domain or variant thereof, a 41BB co-stimulatory domain, an OX40 co-stimulatory domain, a 2B4 co-stimulatory domain, a CTLA-4 co-stimulatory domain, or a combination thereof.

20. The method of claim 19, wherein the co-stimulatory domain comprises any one of SEQ ID NOs: 39-52.

21. The method of claim 1, wherein the CD3ζ co-stimulatory domain comprises SEQ ID NO: 39.

22. The method of claim 1, wherein the CAR comprises SEQ ID NO: 66 or SEQ ID NO: 67 and the ICK comprises SEQ ID NO: 60.
